(19) United States
(12) Patent Application Publication
(10) Pub. No.: US 2023/0154570 A1
(43) Pub. Date: May 18, 2023

(54) DATA PROCESSING SYSTEM FOR PROCESSING GENE SEQUENCING DATA

(71) Applicants: National Yang Ming Chiao Tung University, Hsinchu City (TW); National Taiwan University, Taipei City (TW)

(72) Inventors: Jui-Hung Hung, Tainan City (TW); Chia-Hsiang Yang, New Taipei City (TW); Yi-Chung Wu, Chiayi City (TW); Yen-Lung Chen, Kaohsiung City (TW); Chung-Hsuan Yang, Taipei City (TW)

(73) Assignees: National Yang Ming Chiao Tung University, Hsinchu City (TW); National Taiwan University, Taipei City (TW)

(21) Appl. No.: 17/880,281

(22) Filed: Aug. 3, 2022

(30) Foreign Application Priority Data
Oct. 15, 2021 (TW) 110138325

(51) Int. Cl. G16B 50/00; G16B 30/20; G06F 16/31

(52) U.S. Cl. CPC G16B 50/00; G16B 30/20; G06F 16/316

(57) ABSTRACT

A data processing system can be operated in one of a preprocessing mode, a short-read mapping mode, a sequence assembly mode or a variant calling mode that are related to a to-be-tested DNA sequence. The data processing system includes a sorting engine that supports high-speed processing of sorting in the preprocessing mode and the sequence assembly mode, and a dynamic processing engine that supports dynamic programming calculations in the short-read mapping mode and the variant calling mode. The data processing system may be implemented on a system-on-chip (SoC) for performing accelerated processing of gene sequencing data with reduced memory requirements.

### CROSS-REFERENCE TO RELATED APPLICATION

This application claims priority of Taiwanese Patent Application No. 110138325, filed on Oct. 15, 2021.

### SEQUENCE LISTING

The present application contains a Sequence Listing which has been submitted electronically in XML format and is hereby incorporated by reference in its entirety.

The XML copy, created on Feb. 1, 2023, is named 165455-00101 SL.xml and is 7,187 bytes in size.

### FIELD

The disclosure relates to a data processing system, more particularly to a data processing system for processing gene sequencing data.

### BACKGROUND

In the field of gene sequencing, the next-generation sequencing (NGS) achieves the currently fastest sequencing speed, and is capable of sequencing multiple short gene segments in a parallel processing manner. Accordingly, the NGS may have a processing capacity of a higher order than those of the sequencing techniques based on Sanger sequencing. The applications of NGS are vast and growing, and may facilitate advancement of many biomedical related fields such as Non-Invasive Prenatal Testing (NIPT) and data analysis, recognition of cancer, precise medical diagnosis, biomedical technologies, virus detection, microevolution analysis, etc. As a result, an amount of gene sequencing data to be processed is growing exponentially, and therefore increased time and resource (processing power, memory, etc.) are needed to process and analyze the gene sequencing data.

That is to say, a system that is capable of performing gene sequencing with a higher efficiency and a lower memory requirement is desirable. It is also noted that designing the system in the form of a system on chip (SoC) is also beneficial.

### SUMMARY

Therefore, one object of the disclosure is to provide a data processing system for processing gene sequencing data.

According to one embodiment of the disclosure, the gene sequencing data includes a reference DNA sequence, a plurality of suffix strings, a plurality of indices and a plurality of short-reads. The reference DNA sequence includes characters that represent nitrogen-containing nucleobases. The suffix strings are associated with a reference sequence that includes the reference DNA sequence. Each of the indices indicating a location of the ending character in the reference sequence, and is assigned to a corresponding one of the suffix strings. The short-reads are extracted from a to-be-tested DNA sequence.

The data processing system includes a string generating module, an encoding module that is coupled to the string generating module, a string selecting module that is coupled to the encoding module, a sorting engine that is coupled to the encoding module and the string selecting module, a suffix string array generating module that is coupled to the sorting engine, a data structure generation module that is coupled to the suffix string array generating module, a location generating module; a dynamic processing engine that is coupled to the location generating module, a mapping module that is coupled to the dynamic processing engine and the sorting engine, and a variant calling module that is coupled to the dynamic processing engine.

The data processing system is configured to operate in one of the following modes:

a preprocessing mode, in which
the string generating module is configured to generate a number (N) of partial strings from the suffix strings, respectively, each of the partial strings including first to Kth characters of the respective one of the suffix strings, N being a positive integer greater than 2 and K being a positive integer greater than 2, and N>K,
the encoding module is configured to use binary values to encode the partial strings to generate a number (N) of encoded partial strings, to encode the short-reads to generate a plurality of to-be-tested encoded strings, and to encode the reference DNA sequence to generate a reference encoded string,
the string selecting module is configured to select a number (P*Q) of the encoded partial strings using an upsampling process, and the sorting engine is configured to perform a sorting operation on the number (P*Q) of the encoded partial strings to sort the encoded partial strings in an ascending order, and the string selecting module is configured to select, using a downsampling process, a number (P) of the encoded partial strings from the number (P*Q) of the encoded partial strings that have been sorted as separation strings, wherein P and Q are integers, the sorting engine is configured to perform a grouping operation on the number (N) of the encoded partial strings, using the number (P) of the separation strings, to sort the encoded partial strings into a number (P+1) of groups, and to perform a sorting operation on the encoded partial strings included in each of the number (P+1) of groups, so as to obtain a sorted list of the number (N) of the encoded partial strings,
the suffix string array generating module is configured to generate a suffix string array based on the sorted list of the number (N) of the encoded partial strings, and
the data structure generation module is configured to generate, based on the suffix string array and the associated indices, a data structure associated with the reference DNA sequence, the data structure including a CNT table, an SA table, an F table, an L table and an OCC table, the F table including a column that lists the first characters of the suffix strings included in rows of the suffix string array, the L table including a column that lists the last characters of the suffix strings included in the rows of the suffix string array, the SA table including a column that lists the indices associated with of the suffix strings included in the rows of the suffix string array, the CNT table including a column that lists, for each of the characters, a row address of a prior row immediately before a row at which the character first appears, the OCC table including columns that correspond respectively to the characters and that each list cumulative numbers of appearances of the corresponding one of the characters in the rows of the L table,

a short-read mapping mode, in which
the location generating module is configured to divide each of the short-reads into a plurality of seeds, and, for each of the seeds thus acquired as a result of the division, determine, based on the data structure, at least one candidate row address that is associated with a candidate index indicating a position of the seed in the to-be-tested DNA sequence,
the dynamic processing engine is configured to implement a similarity algorithm with respect to each of the short-reads and the content included in the part of the reference DNA sequence that is indicated by the candidate indices associated with the seeds of the short-reads, so as to obtain a similarity score for the short-read, and
the mapping module is configured to, for each of the short-reads, determine, based on the similarity score, a mapping location for the short-read,

a sequence assembly mode, in which the sorting engine is configured to construct an encoded assembled sequence based on the to-be-tested encoded strings and the reference encoded string and the mapping locations for the short-reads, the encoded assembled sequence indicating a haplotype sequence, and

a variant calling mode, in which
the dynamic processing engine is configured to perform the similarity algorithm with respect to the haplotype sequence and the reference DNA sequence, and
the variant calling module is configured to evaluate a location and a type of a variant in the haplotype sequence based on the result of the similarity algorithm.

### BRIEF DESCRIPTION OF THE DRAWINGS

Other features and advantages of the disclosure will become apparent in the following detailed description of the embodiments with reference to the accompanying drawings, of which:

FIG. 1 is a block diagram illustrating a data processing system according to one embodiment of the disclosure;

FIG. 2 illustrates a reference sequence and a number of suffix strings generated from the reference sequence; FIG. 2 discloses “CATGAAAGGA” as SEQ ID NO: 1.

FIG. 3 illustrates a number of partial strings each generated from a corresponding one of the suffix strings;

FIG. 4 illustrates an exemplary suffix string array that includes a number (N) of suffix strings, and the associated indices; FIG. 4 discloses “CATGAAAGGA” as SEQ ID NO: 1.

FIG. 5 illustrates a data structure associated with a reference DNA sequence, generated based on the suffix string array and the associated indices;

FIG. 6 illustrates a partial data structure in which a partial OCC table and a partial SA table are present;

FIG. 7 is a schematic diagram illustrating an exemplary structure of a sorting engine;

FIG. 8 a schematic diagram illustrating a simplified exemplary structure of one sorting unit of the sorting engine;

FIG. 9 is a circuit diagram illustrating circuitry structures of three sorting units and the connections among them;

FIG. 10 is a block diagram illustrating a dynamic processing engine according to one embodiment of the disclosure;

FIG. 11 is a circuit diagram illustrating circuitry structures of one arithmetic unit of the dynamic processing engine;

FIG. 12 is a circuit diagram partially illustrating the sorting engine performing sorting operation;

FIG. 13 is a circuit diagram partially illustrating the sorting engine performing grouping operations;

FIG. 14 illustrates a similarity algorithm being implemented to construct a scoring matrix; FIG. 14 discloses SEQ ID NO: 1.

FIGS. 15 to 21 are circuit diagrams partially illustrating the sorting engine creating a De Bruijn graph between a reference DNA sequence and a short-read;

FIGS. 22 to 24 are circuit diagrams partially illustrating the sorting engine performing reassembly of an encoded string that corresponds with a to-be-tested DNA sequence;

FIG. 25 illustrates an exemplary reference DNA sequence and a number of short-reads associated with the reference DNA sequence, each having a different mapping location; FIG. 25 discloses SEQ ID NOS. 2-7, respectively, in order of appearance.

FIG. 26 illustrates a similarity score matrix and a scoring direction matrix associated with two strings;

FIG. 27 illustrates a known mathematical model for performing a likelihood calculation; and

FIG. 28 illustrates three exemplary arithmetic units that are configured to perform the likelihood calculations, and to output the corresponding likelihoods.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

Throughout the disclosure, the term “coupled to” or “connected to” may refer to a direct connection among a plurality of electrical apparatus/devices/equipment via an electrically conductive material (e.g., an electrical wire), or an indirect connection between two electrical apparatus/devices/equipment via another one or more apparatus/devices/equipment, or wireless communication.

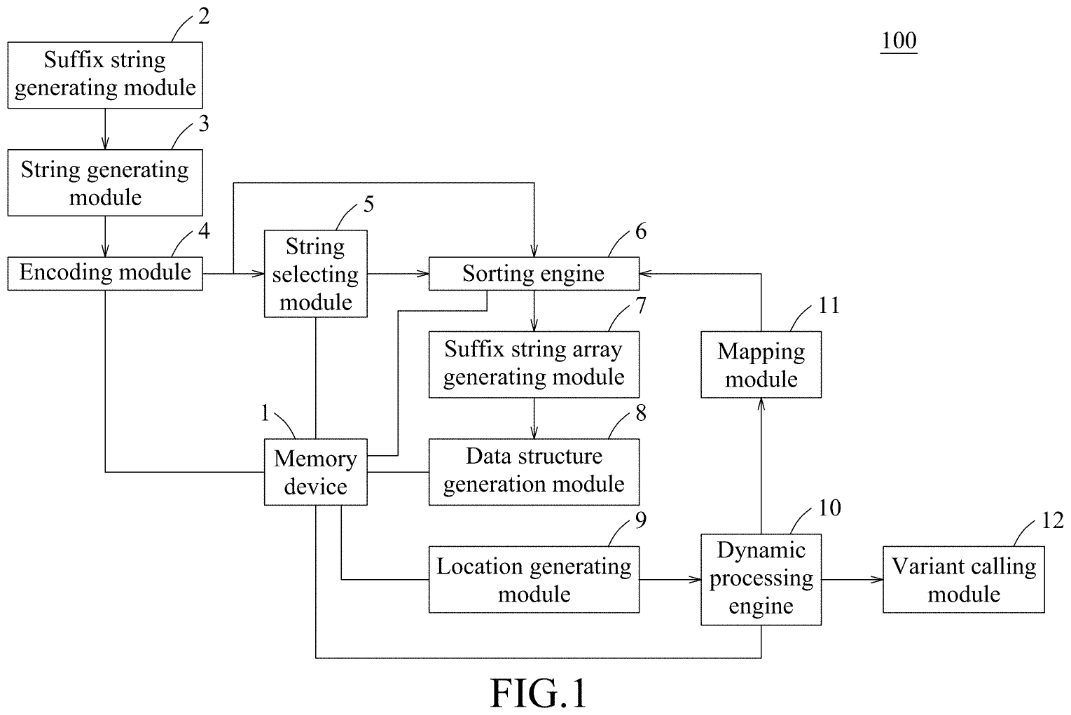

FIG. 1 is a block diagram illustrating a data processing system 100 according to one embodiment of the disclosure. The data processing system 100 in the embodiments is configured to process gene sequencing data.

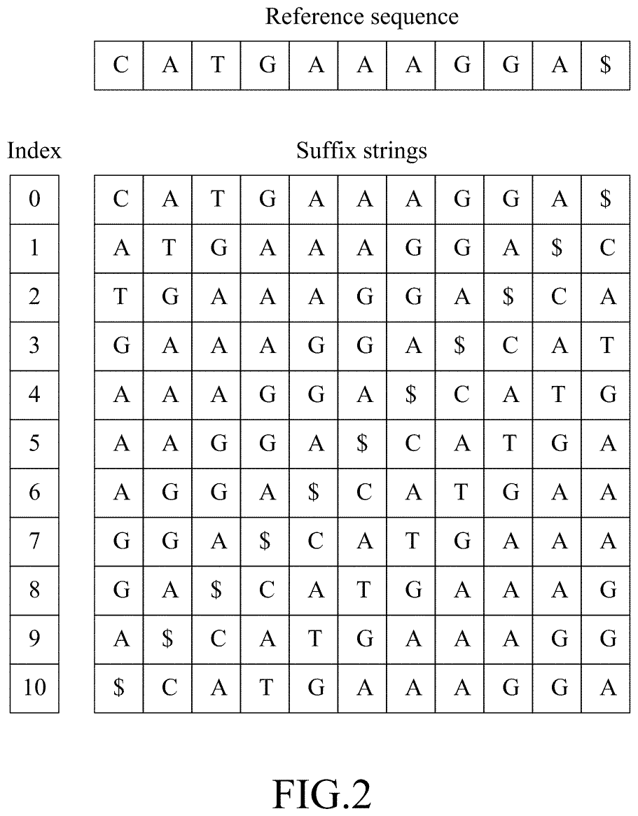

As used herein, the term “gene sequencing data” may refer to segments of data that are associated with a reference DNA sequence (which may be, for example, a human DNA sequence) and a to-be-tested DNA sequence. The gene sequencing data may include a number (N) of suffix strings, a plurality of indices, and a plurality of short-reads extracted from the to-be-tested DNA sequence (as shown in FIG. 2).

In this embodiment, the reference DNA sequence includes a number (N-1) of nitrogenous base characters A, C, G, T that represent four nucleobases (i.e., adenine, cytosine, guanine and thymine), respectively. In use, nucleobases that are not confirmed yet may be represented using one or more characters that are different from the characters mentioned above.

The suffix strings are associated with a reference sequence that has a number N of characters, where N is an integer greater than 2. The reference sequence includes the reference DNA sequence followed by an ending character $ that indicates the end of the reference DNA sequence. The indices indicate relative locations of the N characters in the reference sequence, respectively. Further, the indices are assigned to the suffix strings, respectively (that is to say, each of the indices is made to correspond to one of the suffix strings, as shown in FIG. 2). In this embodiment, the indices may be in the form of integers of 0 to (N-1), but is not limited as such.

An exemplary reference sequence and the associated indices may be represented using the following Table 1. It is noted that the reference sequence (11 characters) includes the reference DNA sequence (SEQ ID NO: 1) (10 characters) and the ending character $ that indicates the end of the reference DNA sequence.

TABLE 1

| Index | 0 | 1 | 2 | 3 | 4 | 5 | 6 | 7 | 8 | 9 | 10 |
|---|---|---|---|---|---|---|---|---|---|---|---|
| Character | C | A | T | G | A | A | A | G | G | A | $ |

The data processing system 100 may be embodied using system on a chip (SoC) structure, and includes a memory device 1, a suffix string generating module 2, a string generating module 3 that is coupled to the suffix string generating module 2, an encoding module 4 that is coupled to the memory device 1 and the string generating module 3, a string selecting module 5 that is coupled to the memory device 1 and the encoding module 4, a sorting engine 6 that is coupled to the memory device 1, the encoding module 4 and the string selecting module 5, a suffix string array generating module 7 that is coupled to the sorting engine 6, a data structure generation module 8 that is coupled to the memory device 1 and the suffix string array generating module 7, a location generating module 9 that is coupled to the memory device 1, a dynamic processing engine 10 that is coupled to the memory device 1 and the location generating module 9, a mapping module 11 that is coupled to the dynamic processing engine 10 and the sorting engine 6, and a variant calling module 12 that is coupled to the dynamic processing engine 10.

The memory device 1 may be embodied using, for example, random access memory (RAM), read only memory (ROM), programmable ROM (PROM), firmware, flash memory, etc. The memory device 1 is configured to store the gene sequencing data and other information that is generated during operations of the data processing system 100.

Each of the above-mentioned modules and engines 2 to 12 may be embodied in: executable software as a set of logic instructions stored in a machine- or computer-readable storage medium of a memory (e.g., the memory device 1); configurable logic such as programmable logic arrays (PLAs), field programmable gate arrays (FPGAs), complex programmable logic devices (CPLDs), etc.; fixed-functionality logic hardware using circuit technology such as application specific integrated circuit (ASIC), complementary metal oxide semiconductor (CMOS), transistor-transistor logic (TTL) technology, etc.; or any combination thereof.

The suffix string generating module 2 is configured to generate the suffix strings that are associated with the reference sequence.

The string generating module 3 is configured to receive the suffix strings from the suffix string generating module 2, and with respect to each of the suffix strings, generate a partial string from the suffix string. In this embodiment, each of the partial strings may include a number (K) of characters, which may be the first (K) characters of the corresponding one of the suffix strings. In this embodiment, K is an integer greater than 2.

The encoding module 4 is configured to perform encoding on the reference DNA sequence, the short-reads stored in the memory device 1, and the partial strings received from the string generating module 3.

Specifically, in one example, for encoding the partial strings, each of the characters $, A, C, G and T may be encoded using a specific binary value (such as 000, 001, 010, 011, 100, respectively). For encoding the short-reads and the reference DNA sequence (in which the character $is not present), each of the characters A, C, G and T may be encoded using another binary value (such as 00, 01, 10, 11, respectively).

In this manner, the encoding module 4 is also configured to encode the partial strings to respectively generate a number (N) of encoded partial strings that correspond respectively with the indices.

The string selecting module 5 is configured to perform a selection operation to select a plurality of separation strings from the encoded partial strings generated by the encoding module 4, and to store the separation strings in the memory device 1.

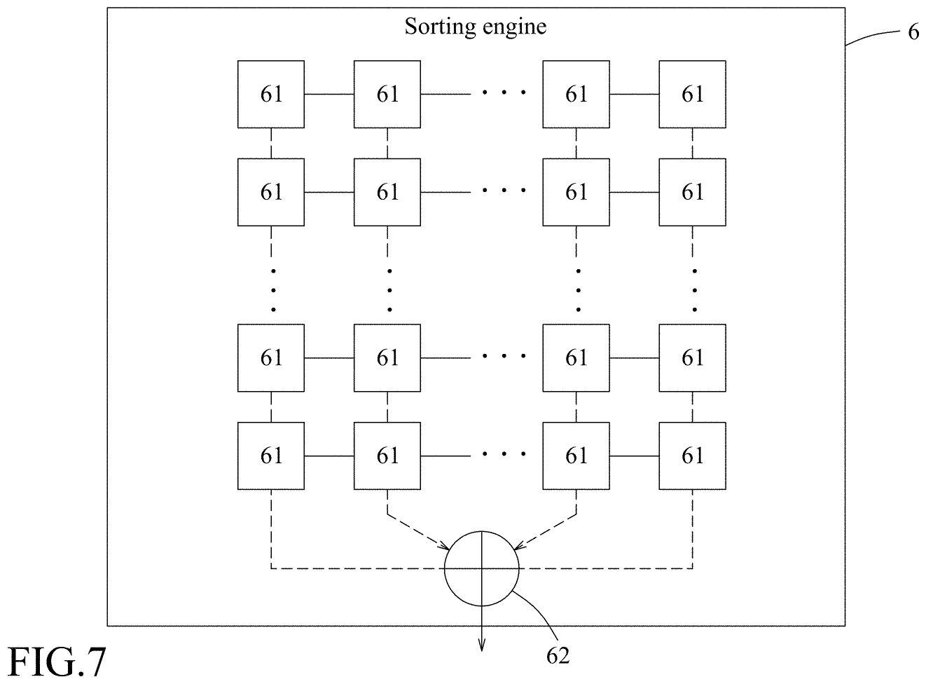

FIG. 7 is a schematic diagram illustrating an exemplary structure of the sorting engine 6.

Specifically, the sorting engine 6 includes a plurality of sorting units 61 that are arranged in a plurality of series connections, and an adder 62 that is connected to each of the sorting units 61.

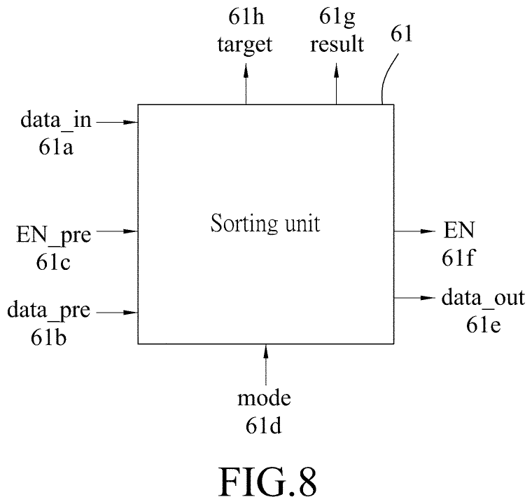

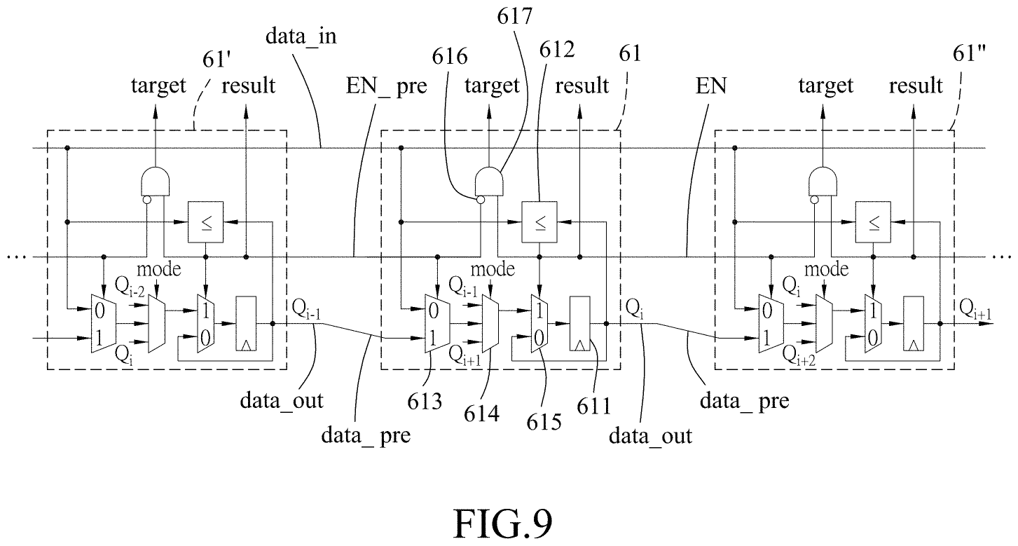

FIG. 8 is a schematic diagram illustrating a simplified exemplary structure of one sorting unit 61. FIG. 9 is a circuit diagram illustrating circuitry structures of three sorting units 61 and the connections among them.

Each sorting unit 61 has a number of input/output nodes. Specifically, as shown in FIGS. 8 and 9, each sorting unit 61 includes a first data input node 61a (data in) for receiving a data signal from other parts of the data processing system 100, a second data input node 61b (data pre) for receiving data from a preceding one of the sorting units 61 that is connected in front of the sorting unit 61 in the same series connection (hereinafter referred to as the preceding sorting unit 61′), a first control input node 61c (EN pre) for receiving a first control signal from the preceding sorting unit 61′, a second control input node 61d (mode) for receiving a second control signal from an external source such as a signal generator circuit, a first output node 61e (data out) for transmitting data to a succeeding one of the sorting units 61 that is connected behind of the sorting unit 61 in the same series connection (hereinafter referred to as the succeeding sorting unit 61″), a second output node 61f (EN) for transmitting the first control signal to the succeeding sorting unit 61″, a third output node 61g (result), and a fourth output node 61h (target).

It is noted that the second data input node 61b (data pre) of the sorting unit 61 is connected to the first output node 61e (data out) of the preceding sorting unit 61′, and the second output node 61f (EN) of the sorting unit 61 is connected to the first control input node 61c (EN pre) of the preceding sorting unit 61′. Similarly, the first output node 61e (data out) of the sorting unit 61 is connected to the second data input node 61b (data pre) of the succeeding sorting unit 61″, and the first control input node 61c (EN pre) of the sorting unit 61 is connected to the second output node 61f (EN) of the preceding sorting unit 61′.

The adder 62 includes a plurality of input nodes that are connected respectively to the third output nodes 61g (result) of the sorting units 61, and an output node that outputs an algebraic sum of inputs received respectively at the input nodes.

The detailed structure of each of the sorting units 61 is shown in FIG. 9. Specifically, each sorting unit 61 includes a register 611, a comparator 612, a first 2*1 multiplexor (MUX) 613, a 3*1 MUX 614, a second 2*1 MUX 615, an inverter 616 and an AND gate 617. Each of the components included in the sorting unit 61 is well known in the related art, and details thereof are omitted herein for the sake of brevity. For the purpose of simpler description, the following description will be made with respect to the sorting unit 61, the preceding sorting unit 61′ and the succeeding sorting unit 61″ as shown in FIG. 9.

The register 611 includes a clock input node (not shown), a data input node, and a data output node that is connected to the first output node 61e (data out) for outputting data stored therein (denoted by Qᵢ) at the first output node 61e (data out). Specially, the clock input nodes of the registers 611 of all sorting units 61 are connected to a same external clock signal generator for receiving the same clock signal.

The comparator 612 includes two input nodes connected to the first data input node 61a (data in) and the data output node of the register 611, respectively, and an output node connected to the third output node 61g (result) and the second output node 61f (EN). The comparator 612 is configured to compare the logic values received by the two input nodes thereof, and to output a logic value “1” when the logic value from the data output node of the register 611 is no smaller than the logic value from the first data input node 61a (data in), and to output a logic value “0” when the logic value from the data output node of the register 611 is larger than the logic value from the first data input node 61a (data in).

The first 2*1 MUX 613 includes a first input node connected to the first data input node 61a (data in), a second input node connected to the second data input node 61b (data pre), a control node connected to the first control input node 61c (EN pre), and an output node connected to the 3*1 MUX 614. When a signal received by the control node of the first 2*1 MUX 613 has a logic value of 0, the logic value of the first input node of the first 2*1 MUX 613 is outputted by the output node; when the signal received by the control node of the first 2*1 MUX 613 has a logic value of 1, the logic value of the second input node of the first 2*1 MUX 613 is outputted by the output node.

The 3*1 MUX 614 includes a first input node connected to the first output node 61e (data out) of the preceding sorting unit 61′, a second input node connected to the output node of the first 2*1 MUX 613, a third input node connected to the first output node 61e (data out) of the succeeding sorting unit 61″, a control node connected to the second control input node 61d (mode), and an output node connected to the second 2*1 MUX 615. Based on the second control signal (e.g., a two-bit signal) received from the second control input node 61d (mode) at the control node of the 3*1 MUX 614, the logic value of one of the first to third input nodes of the 3*1 MUX 614 is outputted at the output node of the 3*1 MUX 614, or a high-impedance state of the 3*1 MUX 614 may be invoked, in which a high impedance is formed between each of the first to third input nodes and the output node of the 3*1 MUX 614.

The second 2*1 MUX 615 includes a first input node connected to the output node of the 3*1 MUX 614, a second input node connected to the output node of the register 611, a control node connected to the output node of the comparator 612, and an output node connected to the input node of the register 611. When a signal (the output of the comparator 612) received by the control node of the second 2*1 MUX 615 has a logic value of 0, the logic value of the first input node of the second 2*1 MUX 615 is outputted by the output node of the second 2*1 MUX 615; when the signal received by the control node of the second 2*1 MUX 615 has a logic value of 1, the logic value of the second input node of the second 2*1 MUX 615 is outputted by the output node of the second 2*1 MUX 615.

The inverter 616 is connected to the first control input node 61c (EN pre), and is configured to implement logical negation on the first control signal received from the first control input node 61c (EN pre) so as to output an opposite logic value of the first control signal.

The AND gate 617 includes two input nodes connected to the inverter 616 and the output node of the comparator 612, respectively, and an output node connected to the fourth output node 61h (target).

It is noted that by adjusting the second control signal and the connections of the components in the sorting units 61, the sorting engine 6 may be configured to perform a number of different operations that will be described in the following paragraphs.

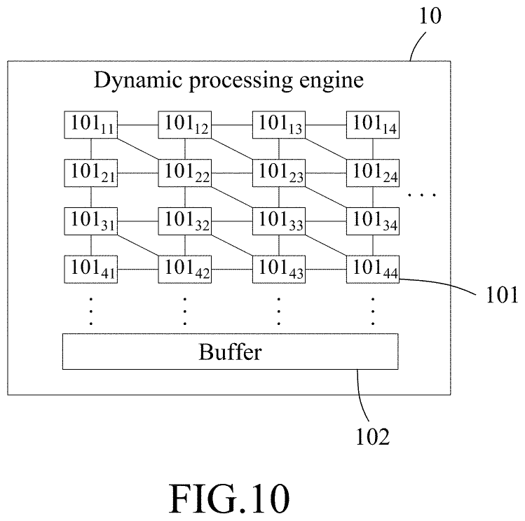

FIG. 10 is a block diagram illustrating the dynamic processing engine 10 according to one embodiment of the disclosure. The dynamic processing engine 10 includes a plurality of operating units 101 that are arranged in a matrix and that are configured to perform the Smith-Waterman algorithm, and a buffer 102 connected to the operating units 101 for storing data outputted by the operating units 101.

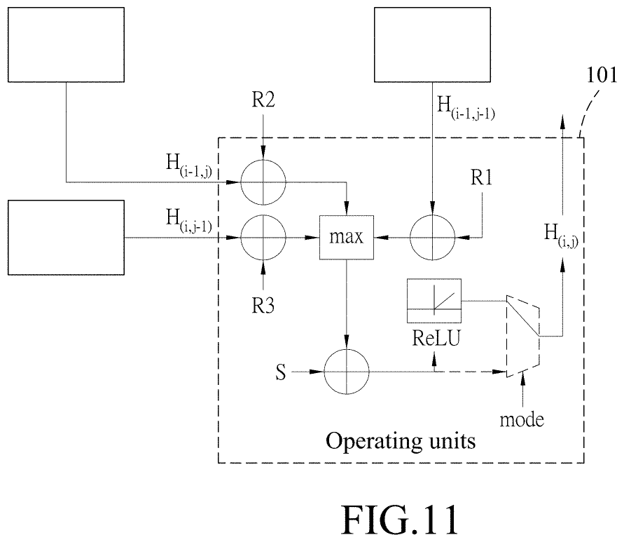

FIG. 11 is a circuit diagram illustrating circuitry structures of one operating unit 101.

The operating unit 101 includes three signal input nodes 101a, 101b and 101c for receiving three input signals (denoted as H_{(i-1, j-1)}, H_{(i-1, j)} and H_{(i, j-1)}), respectively, four parameter input nodes 101d, 101e, 101f and 101g for receiving four parameter inputs (denoted as T1, T2, T3 and S), respectively, a control input node 101h for receiving a control signal (mode) from the second control input node 61d, and an output node 101i for outputting an output signal (denoted as H_{(i, j)}).

Each of the three input nodes 101a, 101b and 101c may be connected to the output node 101i of another operating unit 101. Specifically, the input node 101a of one operating unit 101 may be connected to the output node 101i of an upper one of the operating units 101 arranged directly above said one operating unit 101 in the matrix, the input node 101b of one operating unit 101 may be connected to the output node 101i of an upper-left one of the operating units 101 arranged at an upper-left location to said one operating unit 101 in the matrix, and the input node 101c of one operating unit 101 may be connected to the output node 101i of a left one of the operating units 101 arranged directly to the left of said one operating unit 101.

The operating unit 101 further includes four adders, a rectified linear unit (ReLU), a comparator and a 2*1 MUX. In use, the operating unit 101 is configured to perform the following function:

$${H}_{( i , j )} = max \{ \begin{matrix}( {H}_{( i - 1 , j - 1 )} + R 1 ) + S \\ ( {H}_{( i - 1 , j )} + R 2 ) + S \\ ( H {(}_{i , j - 1 )} + R 3 ) + S \\ 0\end{matrix}$$

where R1, R2, R3 and S represent the received parameters. Since the circuitry structure capable of performing the Smith-Waterman algorithm is well known in the related art, details thereof are omitted herein for the sake of brevity.

Aside from the memory device 1, the sorting engine 6 and the dynamic processing engine 10, each of the modules of the data processing system 100 as described above may be embodied using one or more processors executing one or more software applications. The one or more software applications include instructions that, when being executed by the one or more processors, cause the one or more processors to perform the operations of the modules as described below.

In use, the data processing system 100 is configured to operate in one of the following four different modes: a preprocessing mode; a short-read mapping mode; a sequence assembly mode; and a variant calling mode.

Firstly, in response to receipt of the reference sequence and the indices (from an external source or the memory device 1), the data processing system 100 operates in the preprocessing mode.

Specifically, using the reference sequence and the indices of Table 1 as an example, the suffix string generating module 2 is configured to generate a number (N) of suffix strings (i.e., eleven suffix strings in this example), and to assign an index to each of the suffix strings. Each of the suffix strings starts with a character that is identical to a character in a corresponding location of the reference sequence. For example, the first one of the suffix strings starts with the first character (C) of the reference sequence, and is assigned the index of “0” that corresponds to the first character (C) of the reference sequence; the fifth one of the suffix strings starts with the fifth character (A) of the reference sequence, and is assigned the index of “4” that corresponds to the fifth character (A) of the reference sequence. In this embodiment, each of the suffix strings is generated by shifting the reference sequence for a predetermined number of times. For example, the first one of the suffix strings (with an assigned index of 0) is generated by performing zero shift operations (i.e., identical to the reference sequence), and the second one of the suffix strings (with an assigned index of 1) is generated by performing one shift operation (such that the first character “C” is moved to the end of the suffix string, and all other characters are moved one character ahead). An exemplary result of the suffix strings generated in this manner is shown in FIG. 2.

Afterward, the string generating module 3 generates a number (N) of partial strings from the suffix strings, respectively. Specifically, each of the partial strings includes first to Kth characters of the corresponding one of the suffix strings.

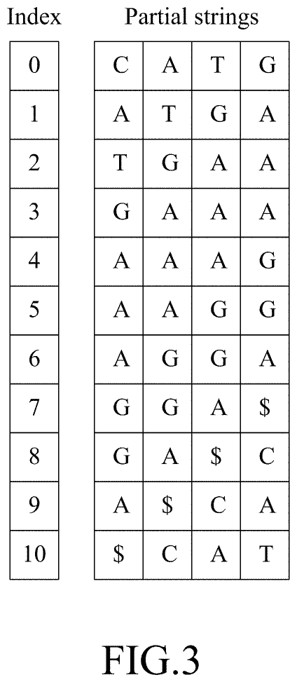

In an example shown in FIG. 3, each of the partial strings includes the first four characters of the corresponding one of the suffix strings (i.e., K=4, and N>K). However it is noted that in other embodiments, the number of (K) may be changed based on the number of (N) and a storage size of the memory device 1. For example, in the case where N is approximately 3*109, the number (K) may be set at 16. In this manner, the amount of data to be stored in the memory device 1 for subsequent processing may be significantly reduced.

Afterward, the encoding module 4 is configured to encode the partial strings to generate a number (N) of encoded partial strings, in a manner as described above. The encoded partial strings corresponds with the indices of the suffix strings, respectively. That is to say, one of the suffix strings and a corresponding one of the encoded partial strings, which is generated from one of the partial strings corresponding to said one of the suffix strings, have the same index. Also, the encoding module 4 is configured to encode the short-reads to generate a plurality of to-be-tested encoded strings, to encode the reference DNA sequence to generate a reference encoded string, and to store the to-be-tested encoded strings and the reference encoded string in the memory device 1.

Afterward, the string selecting module 5 is configured to perform a selection operation to select a plurality of separation strings from the encoded partial strings generated by the encoding module 4, and to store the separation strings in the memory device 1.

Specifically, the string selecting module 5 selects a number (P*Q) of the encoded partial strings, using an upsampling process. The numbers P and Q may be integers. In actual use, the number (N) may be a very large number, and two smaller numbers P and Q are selected such that P*Q«N.

Afterward, the sorting engine 6 is configured to perform a sorting operation on the number (P*Q) of the encoded partial strings to sort the number (P*Q) of the encoded partial strings in an ascending order.

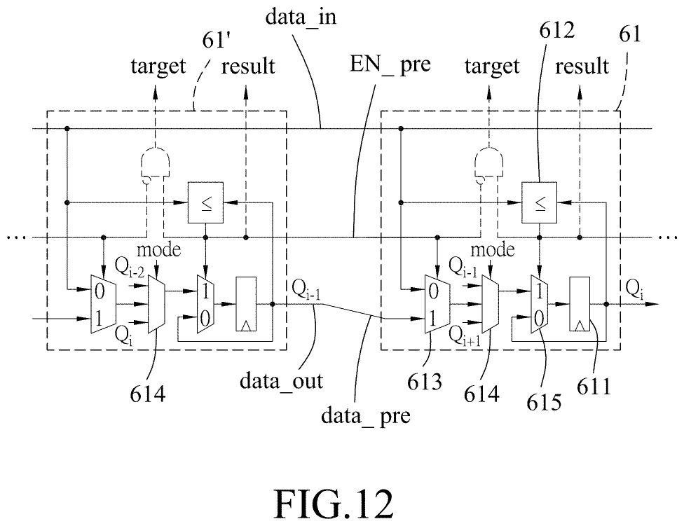

FIG. 12 is a circuit diagram partially illustrating the configuration of the sorting engine 6 performing the sorting operation. Specifically, one sorting unit 61 and a preceding sorting unit 61′ that is connected in front of the sorting unit 61 are present in FIG. 12. In this configuration, the 3*1 MUX 614 is configured to establish a connection between the third input node and the output node thereof. That is, the signal received from the third input node may be directly fed to the output node.

In use, the number (P*Q) of the encoded partial strings will be fed into the sorting units 61 via the first data input nodes 61a. In turn, after a number of clock cycles, the encoded partial string with the smallest encoded binary value may be outputted by the sorting engine 6 first, and the encoded partial string with the largest encoded binary value may be outputted by the sorting engine 6 last; that is, for each encoded partial string, the smaller the encoded binary value thereof, the higher the priority of outputting the encoded partial string.

Afterward, the string selecting module 5 is configured to select a number (P) of the encoded partial strings as the separation strings from the number (P*Q) of the encoded partial strings that have been sorted by the sorting engine 6, using a downsampling process. For example, among the encoded partial strings as shown in FIG. 3, the encoded partial string “CATG” (with the assigned index 0) may be selected as a first one of the number (P) of the encoded partial strings, followed by the encoded partial string “AAGG” (with the assigned index 5) that is selected as a second one of the number (P) of the encoded partial strings. The number (P) of the selected separation strings are then stored in the memory device 1. It is noted that the above-mentioned operations of the string selecting module 5 and the sorting engine 6 involve both the upsampling process and the downsampling process, and therefore, the selected separation strings may be more evenly distributed with respect to the binary values. This may in turn decrease the complexity of the subsequent processes.

Afterward, the sorting engine 6 is configured to perform a grouping operation on the number (N) of the encoded partial strings, using the number (P) of the encoded partial strings (i.e., the separation strings) to sort the encoded partial strings into a number of (P+1) groups.

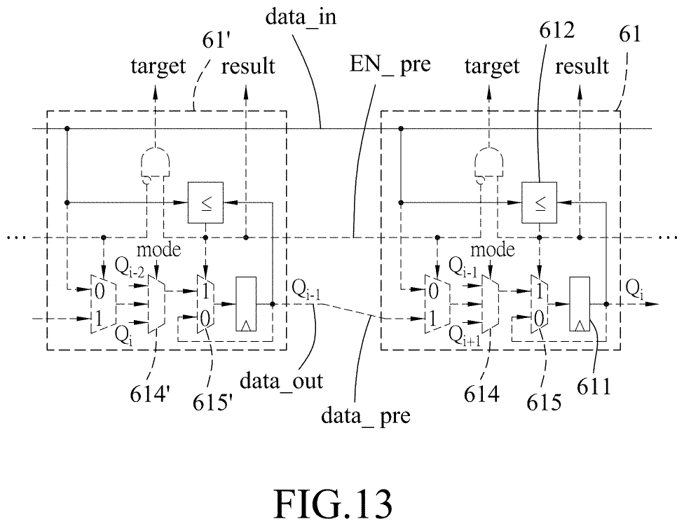

FIG. 13 is a circuit diagram partially illustrating the configuration of the sorting engine 6 performing the grouping operations. Specifically, in the grouping operation, a number (P) of the sorting units 61 operate in the configuration as shown in FIG. 13. In this configuration, for each of the number (P) of the sorting units 61, the 3*1 MUX 614 is controlled by the second control signal to operate in the high-impedance mode, and in turn, the second 2*1 MUX 615 is cutoff.

Specifically, the number (P) of the separation strings are first stored in the registers 611 of the number (P) of the sorting units 61, respectively. In use, the number (N) of the encoded partial strings will be fed into the sorting units 61 via the first data input nodes 61a one by one. Then, the sorting engine 6 is configured to group the encoded partial string, which was fed into the sorting engine 6, into one of the (P+1) groups based on a resulting output of the adder 62. In one example, when the resulting output of the adder 62 is the value 2, the fed encoded partial string may be grouped into a first group. When the resulting output of the adder 62 is the value 1, the fed encoded partial string may be grouped into a second group. When the resulting output of the adder 62 is the value 0, the fed encoded partial string may be grouped into a third group.

Subsequently, after all of the number (N) of the encoded partial strings have been grouped, the sorting engine 6 is configured to perform the sorting operation on the encoded partial strings included in each of the (P+1) groups, so as to obtain a sorted list of the number (N) of the encoded partial strings.

It is noted that the sorting operation may be performed by the sorting engine 6 in the manner as described in the previous paragraphs, using a configuration as shown in FIG. 12. Also, since the number of encoded partial strings included in each of the groups is far less than the number (N), the complexity for sorting the number (N) of the encoded partial strings is also reduced.

Afterward, the suffix string array generating module 7 is configured to generate a suffix string array, based on the sorted list of the number (N) of the encoded partial strings.

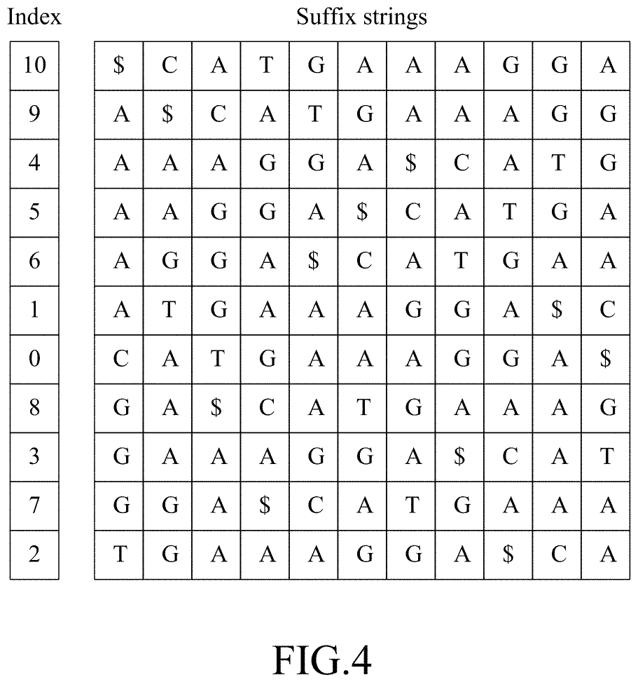

FIG. 4 illustrates an exemplary suffix string array that includes the number (N) of suffix strings (originally from FIG. 2), and the associated indices.

In this suffix string array, the number (N) of suffix strings are sorted using the first characters. It is noted that in this example, the partial suffix strings contain four characters each, and the first three characters are sufficient to obtain a complete sorted list of the number (N) of the encoded partial strings.

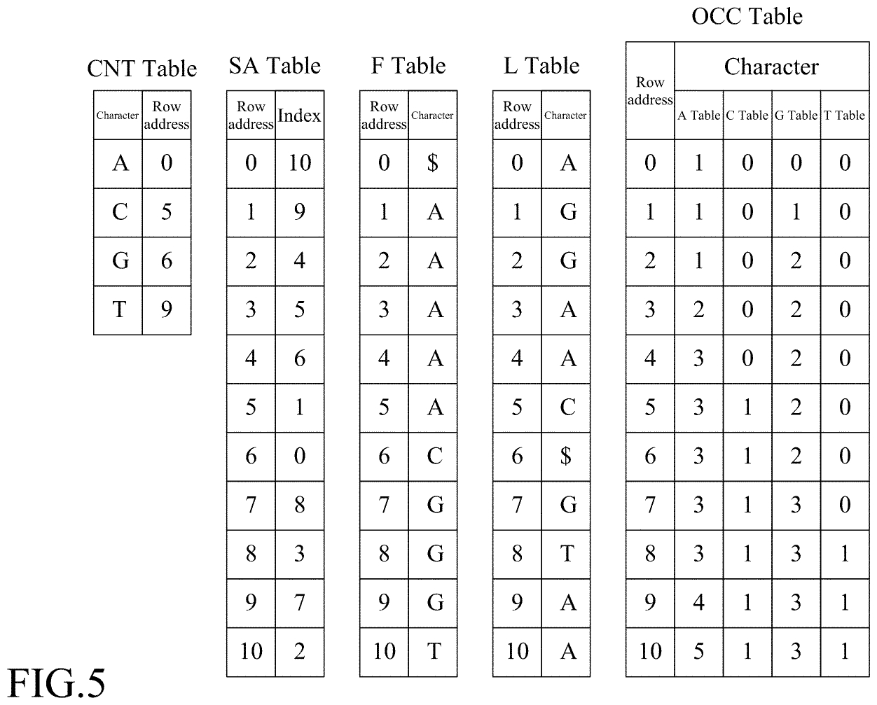

Afterward, the data structure generation module 8 is configured to generate a data structure associated with the reference DNA sequence, based on the suffix string array and the associated indices. Specifically, the data structure may be an FM-index data structure as shown in FIG. 5, and includes a CNT table, an SA table, an F table, an L table and an OCC table.

Specifically, using the suffix string array in FIG. 4 as an example, the F table includes a column that lists the first character of each of the suffix strings included in the rows of the suffix string array. The L table includes a column that lists the last character of each of the suffix strings included in the rows of the suffix string array. The SA table includes a column that lists the index associated with each of the suffix strings included in the rows of the suffix string array. The CNT table includes a column that lists, for each of the characters A, C, G and T, a row address of a prior row immediately before a row, in which the character first appears (for example, the character A first appears in a row address 1, and the prior row address 0 for the character A is included in the CNT table).

The OCC table includes four columns that correspond respectively to the characters A, C, G and T, and each column lists cumulative numbers of appearances of the corresponding one of the characters A, C, G and T in the rows of the L table. That is to say, in each column of the OCC table, each item reflects a number of times of appearance of the corresponding one of the characters from a top item to the corresponding item in the L table.

For example, the character A appears in row addresses 0, 3, 4, 9 and 10 of the L table, and the first three entries (corresponding to the row addresses 0-2) of the A column of the OCC table are each the number 1 indicating that the character A appears only one time from the first to third rows of the L table, the fourth entry (corresponding to the row address 3) of the A column is the number 2 indicating that the character A appears two times, cumulatively from the first to fourth rows of the L table, and the fifth entry (corresponding to the row address 4) of the A column is the number 3 indicating that the character A appears three times, cumulatively from the first to fifth rows of the L table. The data structure thus generated is then stored in the memory device 1.

It is noted that in some embodiments where the memory device 1 has sufficient storage capacity, the entirety of the data structure may be stored in the memory device 1. In other embodiments, since the content of the OCC table is derived from the F table, and the SA table is derived directly from the suffix string array, it may not be necessary to store all of the information contained in the data structure in the case that the storage capacity of the memory device 1 is a concern.

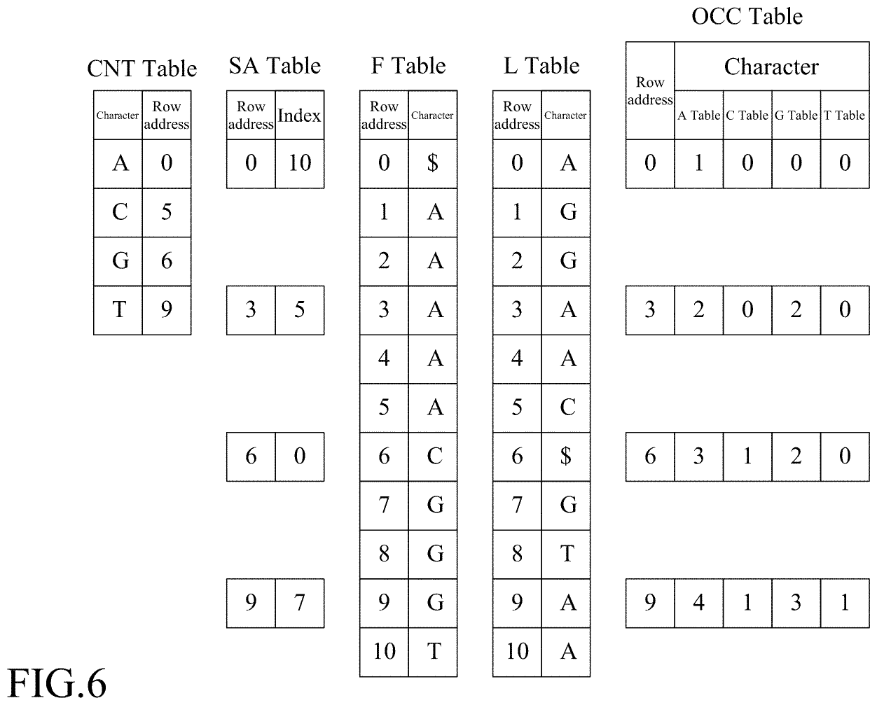

For example, FIG. 6 illustrates a partial data structure in which a partial OCC table and a partial SA table are present, obtained by the data structure generation module 8 using the upsampling or the downsampling to select only a part of the entries from the original data structure of FIG. 5. In this example, the partial OCC table and the partial SA table are constructed using downsampling to obtain one of every three entries in the corresponding original tables. In this manner, the partial data structure contains less information and takes up less storage space of the memory device 1.

Afterward, the data processing system 100 may operate in the short-read mapping mode.

In use, the location generating module 9 is configured to first divide each of the short-reads stored in the memory device 1 into a plurality of seeds. Then, the location generating module 9 is configured to, for each of the seeds thus obtained, determine at least one candidate row address that is associated with a candidate index indicating a position of the seed in the to-be-tested DNA sequence, based on the data structure or the partial data structure.

It is noted that in the case that the partial data structure is stored in the memory device 1, the location generating module 9 is configured to access the memory device 1 to obtain the partial data structure, and to reconstruct the data structure based on the partial data structure before implementing the determination. Specifically, the OCC table may be reconstructed using the partial OCC table and the L table, and the SA table may be reconstructed using the partial SA table, the CNT table and the reconstructed OCC table.

The determination may be done using an index algorithm related to a backward search technique. The index algorithm may be implemented and iterated for a number of times using the following equations:

S[i]=S_{(M-i)+1,}i=1,2, . . . ,M

indexₘᵢₙ[i]=CNT[S[i]]+OCC[indexₘᵢₙ[i−1]−1,S[i]]+1

indexₘₐₓ[i]=CNT[S[i]]+OCC[indexₘₐₓ[i−1]−1,S[i]]

where the seed is to be represented by “S₁S₂ . . . S_{M}” in which S₁, S₂ . . . S_{M} represent individual characters included in the seed, S[i] represents a target character that is to be searched in an i^{th} iteration, indexₘᵢₙ[i] represents a minimum index associated with a row address in which the target character may possibly be located and has an initial value of 0, indexₘₐₓ[i] represents a maximum index associated with a row address in which the target character may possibly be located and has an initial value of N-1, CNT[S[i]] represents a value included in the CNT table associated with the character S[i], and OCC[indexₘᵢₙ[i−1]−1,S[i]] represents a value included in the OCC table with the index address of indexₘᵢₙ[i−1]−1 and associated with the column of the character S[i].

In one example, a short-read having four characters “CATG” may be processed as follows. Firstly, the short-read is divided into a plurality of seeds (e.g., two seeds “CA” and “TG”).

Regarding the seed “CA”, a first iteration yields S[1]=A, indexₘᵢₙ[1]=CNT[A]+OCC[indexₘᵢₙ[0]−1,A]+1 and indexₘₐₓ[1]=CNT[A]+OCC[indexₘₐₓ[0], A]. From the CNT table and the OCC table, it is noted that CNT[A]=0, OCC[indexₘᵢₙ[0]−1,A]=OCC[−1,A]=0 which is a default value, and OCC[indexₘₐₓ[0], A]=OCC[10, A]=5. As such, indexₘᵢₙ[1]=1, and indexₘₐₓ[1]=5.

Then, a second iteration yields S[2]=C, indexₘᵢₙ[2]=CNT[C]+OCC[indexₘᵢₙ[1]−1, C]+1 and indexₘₐₓ[2]=CNT[C]+OCC[indexₘₐₓ[1], C].

From the CNT table and the OCC table, it is noted that CNT[C]=5 OCC[indexₘᵢₙ[1]−1, C]=OCC[0, C]=0 and OCC [indexₘₐₓ[1], C]=OCC [5, C]=1. As such, indexₘᵢₙ[2]=6, and indexₘₐₓ[2]=6.

Since the minimum index and the maximum index have reached convergence, the location generating module 9 may determine the row address “6” as the candidate row address associated with a candidate index of the seed “CA” in the to-be-tested DNA sequence. In other examples, additional iterations may be implemented to determine the candidate row address.

Looking up the SA table obtains SA[6]=0 which is the candidate index. Using the above manner, the seed “TG” may be processed to obtain the candidate index 2.

The operations may then be repeated for each of the seeds divided from the short-reads. In this manner where the seeds are used for processing, the potential scenario that the entire short-read cannot be properly processed because of defects in one of the seeds may be eliminated.

Afterward, the dynamic processing engine 10 is configured to implement a similarity algorithm (e.g., the Smith-Waterman algorithm in this embodiment) with respect to each of the short-reads and the content included in the part of the reference DNA sequence, which is represented in the form of a string and is indicated by the candidate indices associated with the seeds extracted from the short-read. This operation is also known as sequence alignment. Subsequently, a similarity score may be obtained.

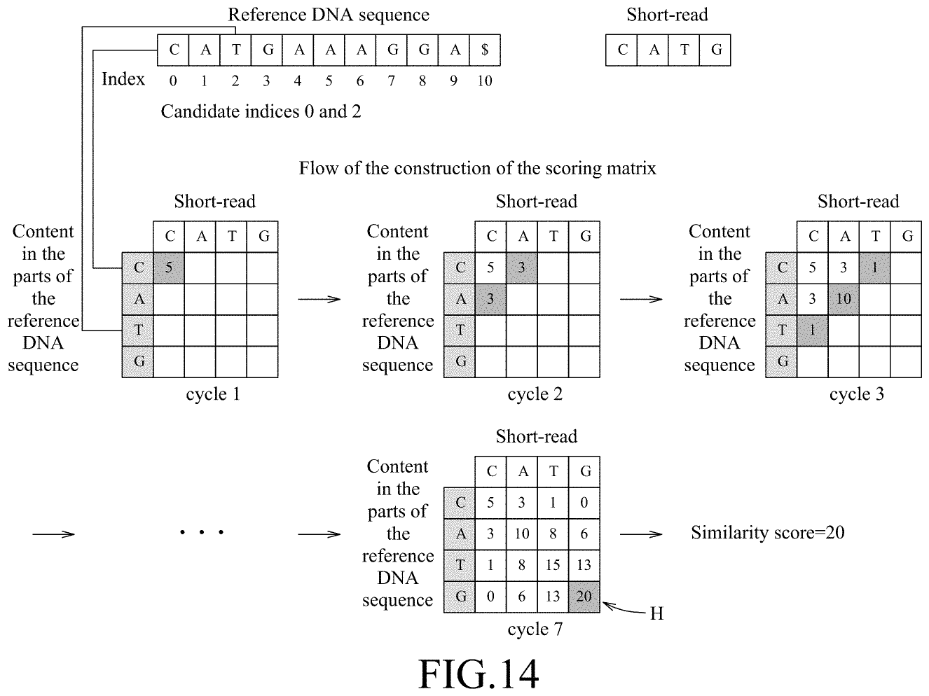

Specifically, as shown in FIG. 14, for the short-read “CATG”, using the two candidate indices 0 and 2, the content included in the parts of the reference DNA sequence is represented in the form of a string “CATG”.

For these two strings, a scoring matrix H is constructed for sequence alignment. A flow of the construction is shown in FIG. 14.

The values of the entries of the scoring matrix H (scores) are determined by comparing one character in the short-read and one character in the string from the reference DNA sequence, and calculating the scores using the following equation:

$${H}_{( i , j )} = max \{ \begin{matrix}( {H}_{( i - 1 , j - 1 )} + T 1 ) + S \\ ( {H}_{( i - 1 , j )} + T 2 ) + S \\ ( H {(}_{i , j - 1 )} + T 3 ) + S \\ 0\end{matrix}$$

where the parameters T1, T2 and T3 are 0 in this example (that is, for example, a logic “0”), and S represents a substitution parameter that is set as a positive value (e.g., 5) in the case that the two characters to be compared are identical, and that is set as a negative value (e.g., −2) in the case that the two characters to be compared are different from each other.

In a first cycle of calculation as shown in FIG. 14, the characters C from both strings are compared, yielding a score of 5 (since the two characters are identical). Such a score is then stored in a corresponding one of the operating units 101 (i.e., 101₁₁).

In a second cycle of calculation as shown in FIG. 14, the character A from each of the two strings is compared with the character C from the other of the two strings, yielding a score of 5−2=3 for either case (since in both cases, the two characters are different from each other). Such scores are then stored in corresponding two of the operating units 101 (i.e., 101₁₂ and 101₂₁).

In a third cycle of calculation as shown in FIG. 14, the characters A from both strings are compared, yielding a score of 5+5=10 (since the two characters are identical). Further, the character T from each of the two strings is compared with the character C from the other of the two strings, yielding a score of 3−2=1 for either case (since in both cases, the two characters are different from each other). Such scores are then stored in corresponding three of the operating units 101 (i.e., 101₁₃, 101₂₂ and 101₃₁).

The dynamic processing engine 10 is configured to continue the calculation until the seventh cycle, where the characters G from both strings are compared, yielding a score of 15+5=20. Such a score is then stored in a corresponding one of the operating units 101 (i.e., 101₄₄). As such, the scoring matrix H is obtained, and a highest score included in the scoring matrix H (i.e., 20) is used as the similarity score associated with the short-read “CATG” and the candidate index 0. The same procedure may then be repeated to obtain another scoring matrix H associated with the seed “TG” of the short-read “CATG” and the candidate index 2 may be obtained, and since the content included in the parts of the reference DNA sequence associated with the candidate index 2 is also represented in the form of the string “CATG”, the similarity score associated with the short-read “CATG” and the candidate index 2 is also 20.

Afterwards, the mapping module 11 is configured to determine, based on the scores stored in the operating units 101, a mapping location for the short-read (e.g., a candidate index associated with a highest similarity score). Using the above calculations, each of the short-reads may be processed to obtain a mapping location.

The data processing system 100 is then switched to operating in the sequence assembly mode.

In use, the sorting engine 6 is configured to construct one or more encoded assembled sequences based on the to-be-tested encoded strings and the reference encoded string stored in the memory device 1, and the mapping location for each of the short-reads. The encoded assembled sequence indicates a haplotype sequence that includes the reference DNA sequence. Specifically, in the cases that no variant is present in the to-be-tested DNA sequence, the corresponding to-be-tested encoded strings and the reference encoded string may be reassembled with only one encoded assembled sequence, which indicates exactly the reference DNA sequence.

In this embodiment, in order to increase the efficiency of constructing the encoded assembled sequence, a De Bruijn graph between the reference DNA sequence and each of the short-reads may first be created. The operations of creating the De Bruijn graphs and constructing the encoded assembled sequence will be described in the following paragraphs.

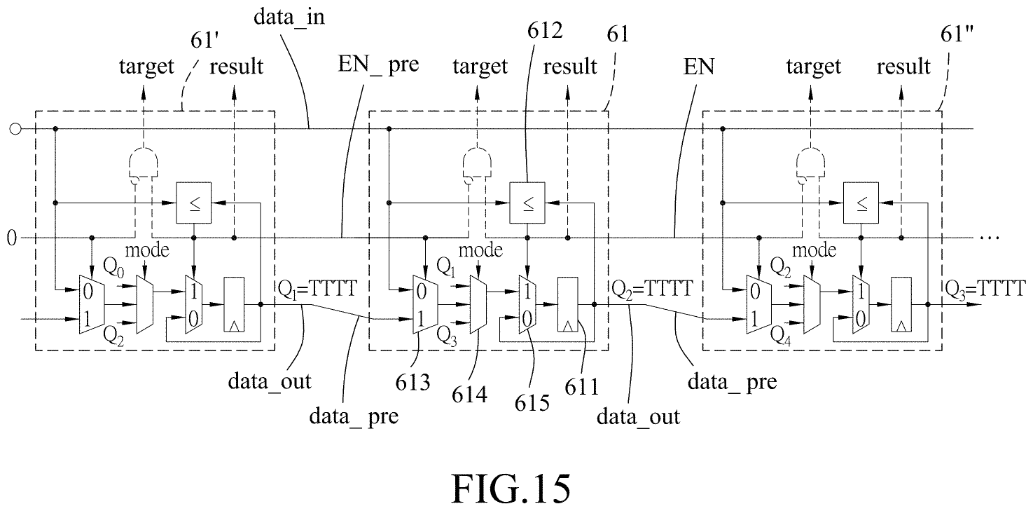

FIG. 15 is a circuit diagram partially illustrating configuration of the sorting engine 6 performing the operation of creating the De Bruijn graphs. Specifically, one sorting unit 61, a preceding sorting unit 61′ that is connected in front of the sorting unit 61 and a succeeding sorting unit 61″ that is connected behind the sorting unit 61 are present in FIG. 15.

In this configuration, the registers 611 are first controlled to store data of a reference encoded sub-sequence. The reference encoded sub-sequence corresponds with a read with consecutive same characters and with a largest binary value. In this example, the read is “TTTT”, and the reference encoded sub-sequence is “11111111”. It is noted that while in the example, the data stored in the registers 611 and outputted by the output nodes thereof (Q1, Q2 and Q3, respectively) are shown in the form of the read (“TTTT”), in use, it is the binary values “11111111” that are actually stored in the registers 611.

Further, for each of the sorting units 61, the first 2*1 MUX 613 of the preceding sorting unit 61′ is configured to have the first input node thereof connected to the output node thereof, and the 3*1 MUX 614 is configured to have the third input node thereof connected to the output node thereof.

In use, for each of the sorting units 61, the first data input node 61a is configured to receive a plurality of encoded sub-sequences. The encoded sub-sequences are associated with consecutive same characters included in the to-be-tested encoded string encoded from the short-reads or the reference encoded string encoded from the reference DNA sequence. In this manner, the encoded sub-sequences are stored in the registers 611 of corresponding ones of the sorting units 61, so as to complete the operation of creating the De Bruijn graphs. Specifically, using the example of FIG. 15, the registers 611 of the sorting units 61 are first controlled to store the data “TTTT”.

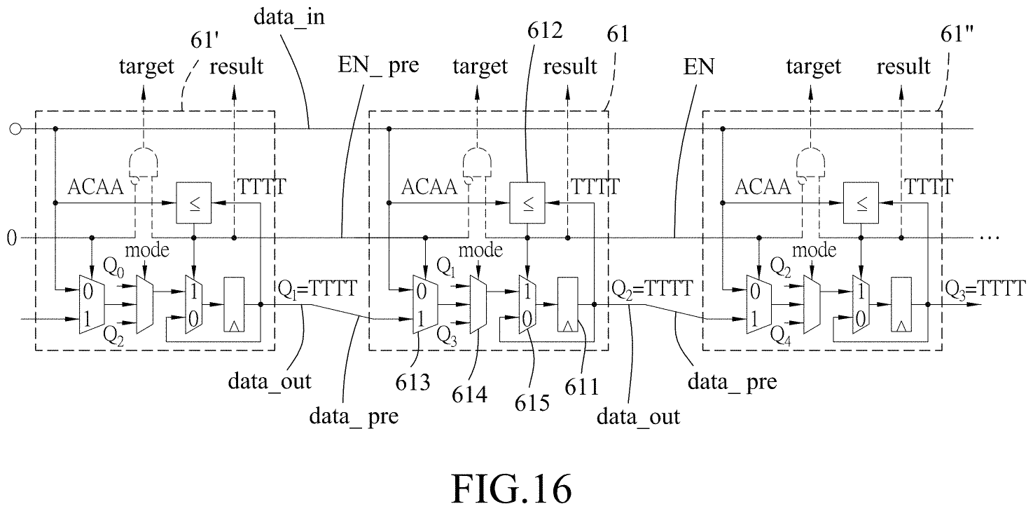

Further referring to FIG. 16, a short-read “ACAATT” (also referred to as a De Bruijn sequence), is to be inputted. The sorting engine 6 is configured such that the encoded sub-sequences that are associated with a 4-character segment contained in the De Bruijn sequence (also known as a first 4-mer) are received by the first data input nodes 61a. For example, the first 4-mer includes the first four characters “ACAA” of the De Bruijn sequence. Then, the comparator 612 of each of the sorting units 61 compares the binary value of one encoded sub-sequence that is associated with the first 4-mer (i.e., “ACAA” (in this case) and the binary value the data “TTTT”.

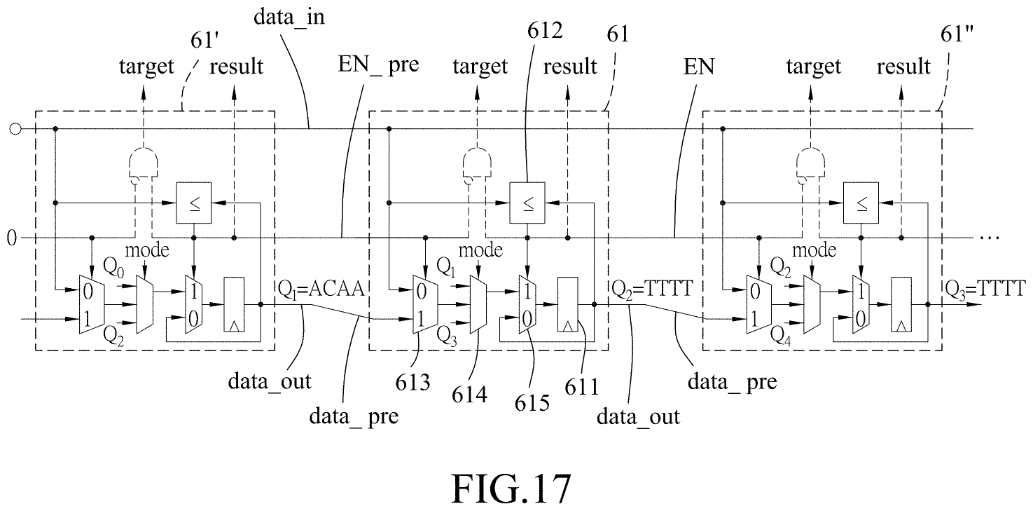

In the case that the binary value of the data stored in the register 611 is larger than that of the encoded sub-sequence, the comparator 612 outputs a digital signal “1” to the control node of the second 2*1 MUX 615. Otherwise, the comparator 612 outputs a digital signal “0” to the control node of the second 2*1 MUX 615. After one clock cycle, the data stored in the register 611 of the preceding sorting unit 61′ becomes the encoded sub-sequence that is associated with the first 4-mer “ACAA” (i.e., Q1 becomes “ACAA”), while the data stored in the register 611 of other sorting units 61 remain unchanged (i.e., Q2 and Q3 are “TTTT”), as shown in FIG. 17.

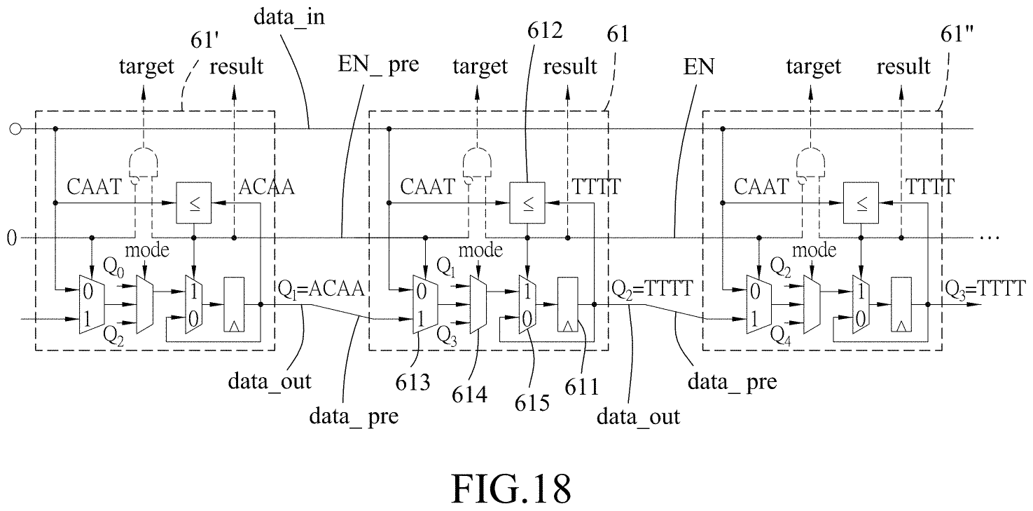

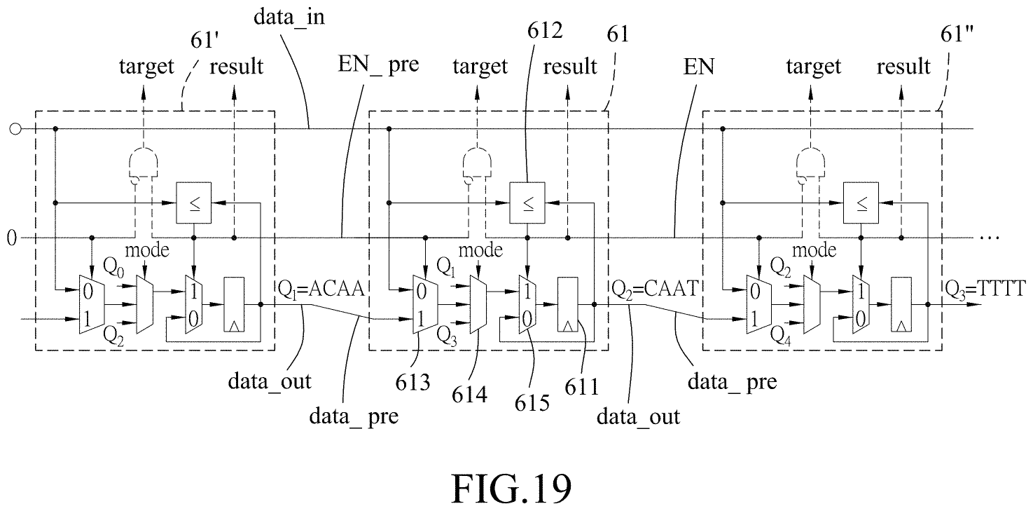

Then, as shown in FIG. 18, the encoded sub-sequences that are associated with a second 4-mer are received by the first data input nodes 61a. For example, the second 4-mer includes the second to fifth characters “CAAT” of the De Bruijn sequence. Then, the comparator 612 of each of the sorting units 61 compares the binary value of one encoded sub-sequence that is associated with the second 4-mer (i.e., “CAAT” in this case) and the binary value of the data stored in the register 611. In this manner, after another clock cycle, the data stored in the register 611 of the sorting unit 61 becomes the encoded sub-sequence that is associated with the second 4-mer “CAAT” (i.e., Q2 becomes “CAAT”), while the data stored in the register 611 of other sorting units 61 remain unchanged (i.e., Q1 remains “ACAA” and Q3 remains “TTTT”), as shown in FIG. 19.

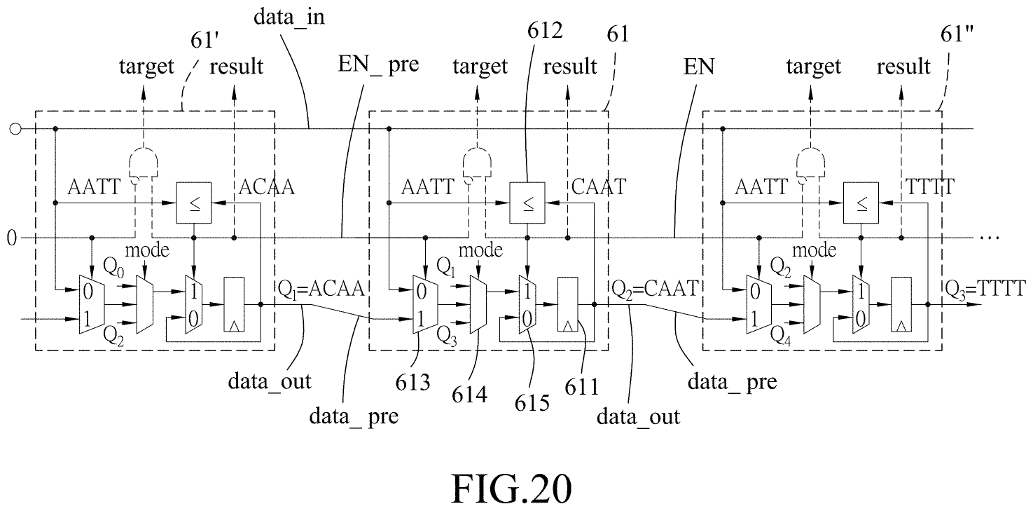

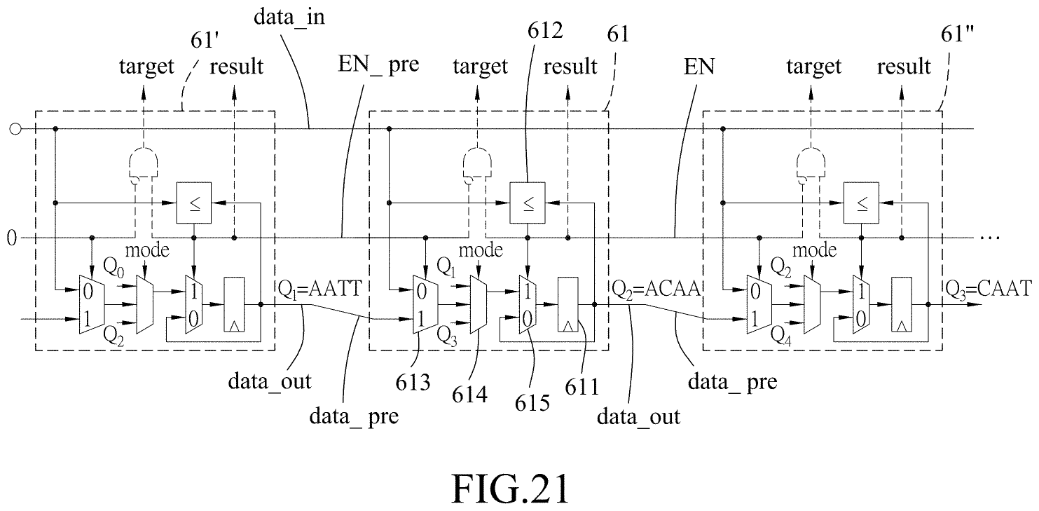

Then, as shown in FIG. 20, the encoded sub-sequences that are associated with a third 4-mer are received by the first data input nodes 61a. For example, the third 4-mer includes the third to sixth characters “AATT” of the De Bruijn sequence. Then, the comparator 612 of each of the sorting units 61 compares the binary value of one encoded sub-sequence that is associated with the third 4-mer (i.e., “AATT” in this case) and the binary value of the data stored in the register 611. In this manner, after another clock cycle, the data stored in the register 611 of the preceding sorting unit 61′ becomes the encoded sub-sequence that is associated with the third 4-mer “AATT” (i.e., Q1 becomes “AATT”), the data stored in the register 611 of the sorting unit 61 becomes the encoded sub-sequence that is associated with the first 4-mer “ACAA” (i.e., Q2 becomes “ACAA”), while the data stored in the register 611 of the succeeding sorting unit 61″ becomes the encoded sub-sequence that is associated with the second 4-mer “CAAT” (i.e., Q3 becomes “CAAT”), as shown in FIG. 21. The operation may then be repeated for other 4-mers until all the encoded sub-sequences associated with the short-read “ACAATT” are stored in the registers 611, thereby completing the construction of the De Bruijn graphs.

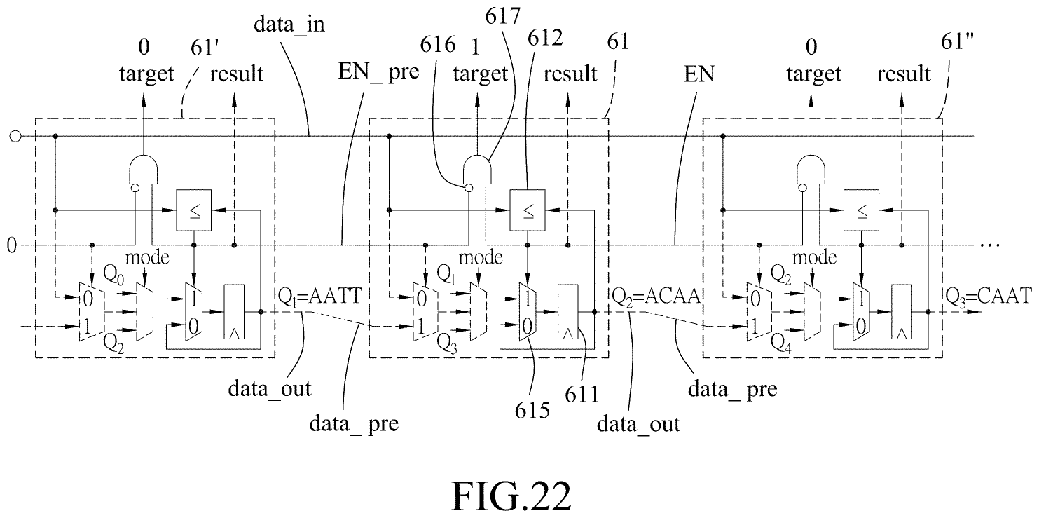

After the De Bruijn graphs have been constructed for the short-reads, when it is intended to reassemble an encoded string that corresponds with the short-reads (that is associated with the to-be-tested DNA sequence), the configuration of the sorting engine 6 as shown in FIG. 22 may be employed.

Specifically, the operations for reassembly of the encoded string that corresponds with the short-reads may be done as follows.

Firstly, an encoded sub-string that is associated with a k-mer (the first to a kth characters of the short-read) of one of the short-reads with a smallest mapping location may be used as an input to the first data input node 61a of each of the sorting units 61. Then, the comparator 612 of each of the sorting units 61 is configured to compare the binary values of the encoded sub-sequence and the encoded sub-string. Subsequently, the fourth output node 61h of one of the sorting units 61 outputs the logic signal “1”, while the fourth output nodes 61h of other sorting units 61 output the logic signal “0”. This results in the data stored in the one of the sorting units 61 being outputted for reassembly of the encoded string that corresponds with the short-read.

Then, an encoded sub-string that is associated with another k-mer (the second, third, . . . , the (k+1)^{th} characters of the short-read) of the one of the short-reads with a smallest mapping location may be used as an input to the first data input node 61a of each of the sorting units 61. Then, the comparator 612 of each of the sorting units 61 is configured to compare the binary values of the encoded sub-sequence and the encoded sub-string. Subsequently, the fourth output node 61h of one of the sorting units 61 outputs the logic signal “1”, while the fourth output nodes 61h of other sorting units 61 output the logic signal “0”. This results in the data stored in the one of the sorting units 61 being outputted for reassembly of the encoded string that corresponds with the short-read. The above operations may then be similarly repeated for other characters of the one of the short-reads, and for characters of other short-reads until the encoded string that corresponds with the short-reads is assembled. Then, the encoded string may be stored in the memory device 1, and in use, the encoded string may be decoded using an inverse operation of the encoding operation to obtain the corresponding haplotype sequence.

Subsequently, another round of the above operations may be repeated sequentially for each of the other short-reads, for example starting with another one of the short-reads with the second smallest mapping location.

FIG. 22 illustrates an exemplary configuration for performing the above operations, using the short-read “ACAATT” as an example. In this configuration, the first 2*1 MUXs 613 and the 3*1 MUXs 614 are controlled in a cutoff mode. It is noted that the goal of the operations is to obtain an encoded string that corresponds with the short-read “ACAATT”.

In use, an encoded sub-string that is associated with a 3-mer ACA (the first three characters of the short-read “ACAATT”) may be used as an input to the first data input node 61a of each of the sorting units 61. As such, the comparator 612 of each of the sorting units 61 is configured to compare the binary values of the encoded sub-sequence and the encoded sub-string. Subsequently, the fourth output node 61h of the sorting unit 61 (whose register 611 stores the data “ACAA” therein) outputs the logic signal “1”, while the fourth output node 61h of each of other sorting units 61 outputs the logic signal “0”. This results in the data “ACAA” being outputted for reassembly of the encoded string that corresponds with the short-read “ACAATT”.

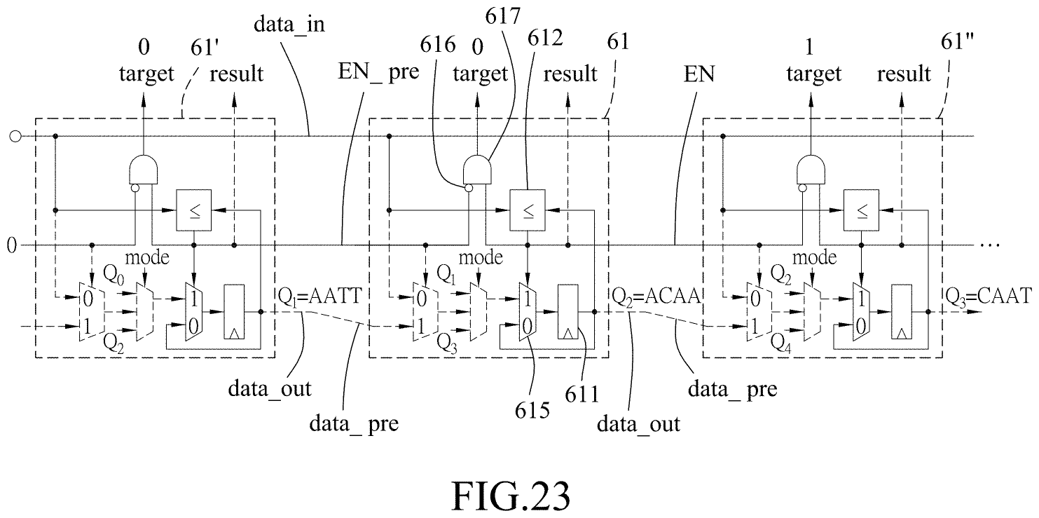

Then, as shown in FIG. 23, an encoded sub-string that is associated with a 3-mer “CAA” (the second, third and fourth characters of the short-read “ACAATT”) may be used as an input to the first data input node 61a of each of the sorting units 61. As such, the comparator 612 of each of the sorting units 61 is configured to compare the binary values of the encoded sub-sequence and the encoded sub-string. Subsequently, the fourth output node 61h of the succeeding sorting unit 61″ (whose register 611 stores the data “CAAT” therein) outputs the logic signal “1”, while the fourth output node 61h of each of other sorting units 61 outputs the logic signal “0”. This results in the data “CAAT” being outputted for reassembly of the encoded string that corresponds with the short-read “ACAATT”. Combining the data obtained in the above two iterations yields the data “ACAAT” for reassembly of the encoded string that corresponds with the short-read “ACAATT”.

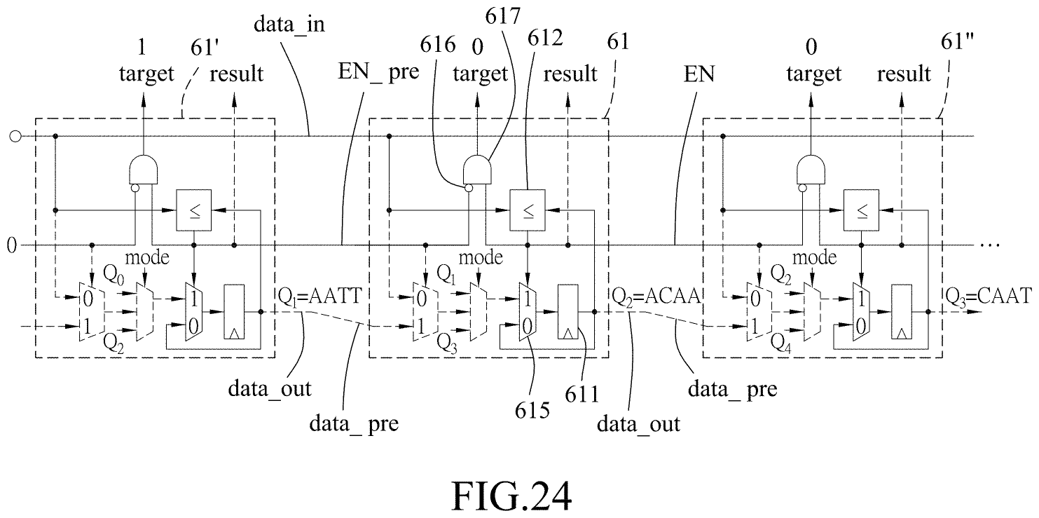

Then, as shown in FIG. 24, an encoded sub-string that is associated with a 3-mer “AAT” (the third, fourth and fifth characters of the short-read “ACAATT”) may be used as an input to the first data input node 61a of each of the sorting units 61. As such, the comparator 612 of each of the sorting units 61 is configured to compare the binary values of the encoded sub-sequence and the encoded sub-string. Subsequently, the fourth output node 61h of the preceding sorting unit 61′ (whose register 611 stores the data “AATT” therein) outputs the logic signal “1”, while the fourth output node 61h of each of other sorting units 61 outputs the logic signal “0”. This results in the data “AATT” being outputted for reassembly of the encoded string that corresponds with the short-read “ACAATT”. Combining the data obtained in the above three iterations yields the data “ACAATT”, which constitutes the encoded string that corresponds with the short-read “ACAATT”.

Using the above operations, the sorting engine 6 is capable of obtaining the encoded string and a haplotype sequence that corresponds with the short-read. The above operations may then be repeated for other short-reads.

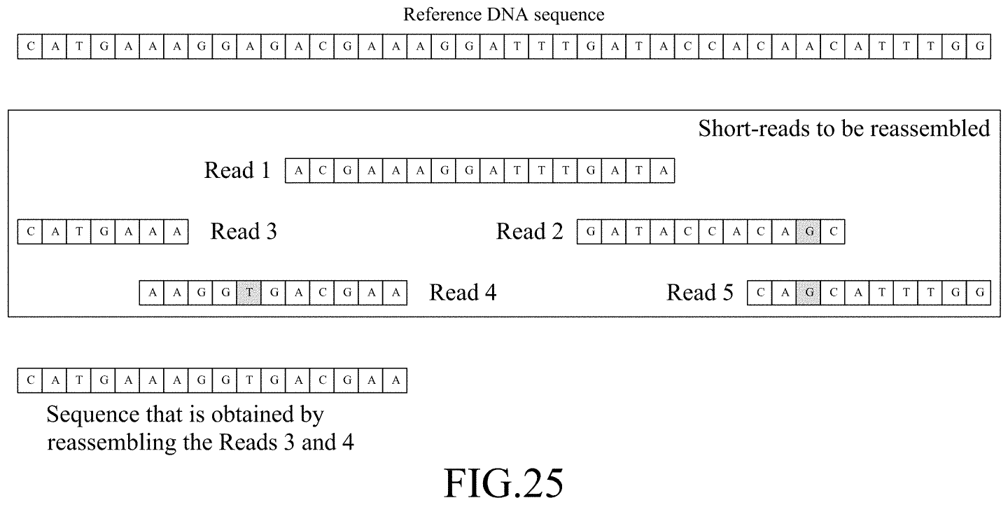

FIG. 25 illustrates an exemplary reference DNA sequence and a number of short-reads that are associated with the reference DNA sequence and that are to be reassembled, each having a different mapping location. In this example, Read 3 has a smallest mapping location, and as such, the sorting engine 6 may be configured to first perform the operations for obtaining the encoded string with respect to Read 3, followed by Read 4, Read 1, Read 2 and Read 5 in said order, so as to obtain a combined encoded string (not depicted in the drawings) that corresponds with the reference DNA sequence. FIG. 25 also illustrates a sequence that is obtained by reassembling the Reads 3 and 4.

Afterward, the data processing system 100 may be configured to operate in the variant calling mode. In the variant calling mode, the data processing system 100 is configured to identify a location of a variant in the haplotype sequence(s) obtained in the above operations, and to estimate a type of the variant.

It is noted that in the example of FIG. 25, some of the short-reads contain variant(s) resulted from, for example, single nucleotide polymorphism (SNP) (i.e., substitution of a single nucleotide at a specific position), as shown in the shaded blocks of FIG. 25.

In the variant calling mode, the dynamic processing engine 10 is configured to perform a similarity algorithm (e.g., the Smith-Waterman algorithm in this embodiment, the operations of which are described in the above paragraphs) with respect to each of the haplotype sequence and the reference DNA sequence (which is represented in the form of a string). This operation is also known as sequence alignment. Subsequently, a similarity score matrix and the corresponding similarity score may be obtained. The details of obtaining the similarity score may be done in a manner similar to those as described before, and are not repeated here for the sake of brevity.

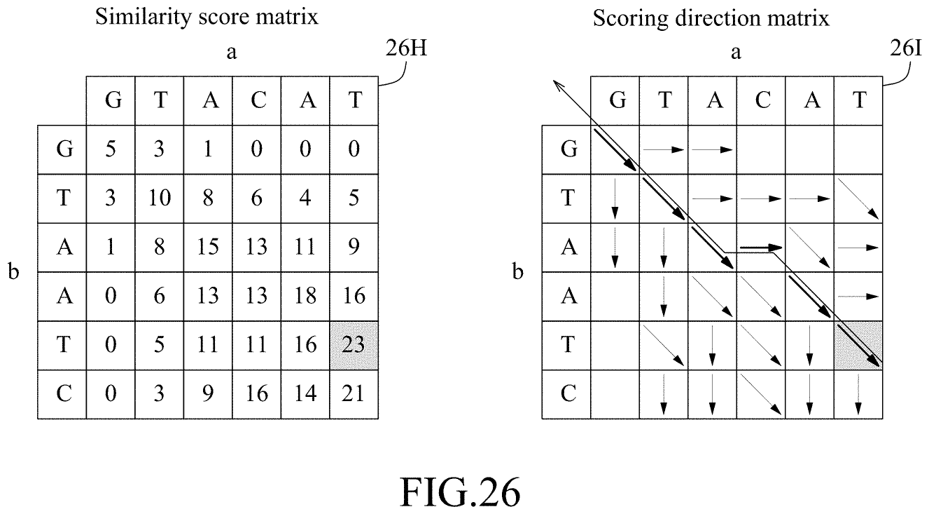

Specifically, as shown in the left portion of FIG. 26, an exemplary reference DNA sequence “GTACAT” and an exemplary haplotype sequence “GTAATC” are to be processed by the dynamic processing engine 10. It is noted that while in this example, each of the haplotype sequence and the reference DNA sequence has a length of 6 characters, in actual implementations, each of the haplotype sequence and the reference DNA sequence may have a length of up to 300 characters, depending on a size of the buffer 102. In this embodiment, the dynamic processing engine 10 obtains a similarity score matrix 26H (shown in the left portion of FIG. 26) and a scoring direction matrix 26I (shown in the right portion of FIG. 26) that contains information related to the similarity score matrix 26H.

In operation, the first characters of the reference DNA sequence “GTACAT” and the exemplary haplotype sequence “GTAATC” are compared. Since the first characters of the two sequences are identical to each other (G), the score in the corresponding entry of the similarity score matrix 26H is 5, and the content in the corresponding entry of the scoring direction matrix is “ [symbol] ” (which is, in actuality, represented using corresponding binary values), meaning that a source of change of the score is from an upper left direction. Then, the second character of the reference DNA sequence and the first character of the exemplary haplotype sequence are compared. Since the two characters are different, the score in the corresponding entry of the similarity score matrix 26H is 5−2=3, and the content in the corresponding entry of the scoring direction matrix is “→”, meaning that a source of change of the score is from a left direction. Then, the first character of the reference DNA sequence and the second character of the exemplary haplotype sequence are compared. Since the two characters are different, the score in the corresponding entry of the similarity score matrix is 5−2=3, and the content in the corresponding entry of the scoring direction matrix is “↓”, meaning that a source of change of the score is from an up direction. Using the above manner, other entries of the similarity score matrix and the scoring direction matrix are filled, as seen in FIG. 26. The above operations may then be repeated for each of the haplotype sequences and the reference DNA sequence, and the resulting similarity score matrices and the scoring direction matrices may be stored in the buffer 102.

Then, for each of the haplotype sequences (that are parts of the to-be-tested DNA sequence), the variant calling module 12 is configured to evaluate a location and a type of a variant (if any) based on the similarity score matrix and the scoring direction matrix. The type of the variant may include, for example, an insertion mutation (IM), a deletion mutation (DM), a single nucleotide polymorphism (SNP), etc.

Specifically, the variant calling module 12 is configured to determine one entry of the similarity score matrix with a highest similarity score (e.g., the shaded entry with the similarity score 23 in the example of FIG. 26), and to determine a backtrack from one entry of the scoring direction matrix that corresponds with the determined one entry of the similarity score matrix (the shaded entry in the example of FIG. 26), going in a route that is indicated by the reverse of the arrows in the similarity score matrix, to a top left entry of the scoring direction matrix (which is represented using boldfaced arrows in the example of FIG. 26).

In this manner, whenever the backtrack includes an entry that is not “ [symbol] ”, it may be determined that a variant exists at the corresponding location of the haplotype sequence (since the character is different from that of the reference DNA sequence). Specifically, a “↓” contained in the backtrack may indicate that an IM is present at the corresponding location of the haplotype sequence, and a “→” contained in the backtrack may indicate that a deletion mutation DM is present at the corresponding location of the haplotype sequence. In some examples, the backtrack may include exclusively “ [symbol] ” without “↓” and “→”, but based on the corresponding similarity scores, appearance of an SNP may be determined.

In the example of FIG. 26, the backtrack contains one entry, on the fourth column, with the direction “→”. That is to say, a deletion mutation may be present in the fourth character of the haplotype sequence. Then, the variant calling module 12 is configured to perform a marking operation to “insert” a dummy character “-” in the fourth character of the haplotype sequence (since it is determined that a deletion mutation is present), to shift the characters after the fourth character backward by one character, and to output the resulting haplotype sequence “GTA-AT”.

In this embodiment, after the variant(s) are identified from the haplotype sequences of the to-be-tested DNA sequence, the dynamic processing engine 10 is further configured to perform a likelihood calculation for each variant.

In use, the likelihood calculation is done based on one or more short-reads that contain one specific variant, one haplotype sequence that contains the one specific variant, and the reference DNA sequence (which is selected to contain no variant), and may be used to determine, with respect to a double stranded (DS)-DNA of the to-be-tested DNA sequence, the likelihoods of: (1) the DS-DNA containing no variant at a location that corresponds with the one specific variant (meaning both of the parents of a test subject of the to-be-tested DNA sequence has no such variant); (2) the DS-DNA containing two variants at a location that corresponds with the one specific variant (meaning both of the parents of the test subject have such variant); and (3) the DS-DNA containing one variant at a location that corresponds with the one specific variant (meaning one of the parents of the test subject has such variant).

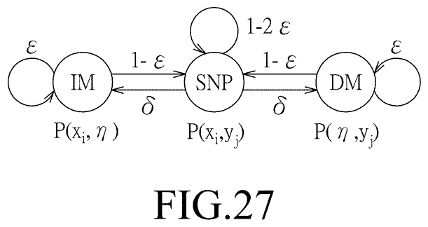

FIG. 27 illustrates a known mathematical model (i.e., Pair-HMM state diagram) for performing such operations. The mathematical model includes the following equations, for two sequences x,y:

$${V}_{S} ( i , j ) = P ( {x}_{i} , {y}_{j} ) \cdot max \{ \begin{matrix}( 1 - 2 δ ) \cdot {V}_{S} ( i - 1 , j - 1 ) \\ ( 1 - ε ) \cdot {V}_{I} ( i - 1 , j - 1 ) \\ ( 1 - ε ) \cdot {V}_{D} ( i - 1 , j - 1 )\end{matrix}$$

$${V}_{I} ( i , j ) = P ( {x}_{i} , η ) \cdot max \{ \begin{matrix}δ \cdot {V}_{S} ( i - 1 , j ) \\ ε \cdot {V}_{D} ( i - 1 , j )\end{matrix} , and$$

$${V}_{D} ( i , j ) = P ( η , {y}_{j} ) \cdot max \{ \begin{matrix}δ \cdot {V}_{S} ( i , j - 1 ) \\ ε \cdot {V}_{D} ( i , j - 1 )\end{matrix}$$

where V_{S}(i,j) represents a likelihood of an occurrence of a variant that is in an i^{th} character of the sequence x with respect to a j^{th} character of the sequence y and that is resulted from SNP, V_{I}(i,j) represents a likelihood of an occurrence of a variant in an i^{th} character of the sequence x with respect to a j^{th} character of the sequence y resulted from IM, V_{D}(i,j) represents a likelihood of an occurrence of a variant in an i^{th} character of the sequence x with respect to a j^{th} character of the sequence y resulted from DM, P(xᵢ,yᵢ) represents a likelihood of an occurrence of a variant in the sequence x with respect to the sequence y resulted from SNP (i.e., a likelihood of the i^{th} character of the sequence x being identical to the j^{th} character of the sequence y), P(xᵢ, η) represents a likelihood of an occurrence of a variant in the sequence x with respect to the sequence y resulted from IM (i.e., a likelihood of the i^{th} character of the sequence x corresponding with an empty base of the sequence y), P(η, yᵢ) represents a likelihood of an occurrence of a variant in the sequence x with respect to the sequence y resulted from DM (i.e., a likelihood of an empty base of the sequence x corresponding with the j^{th} character of the sequence y), and δ and ε are predetermined parameters.

Applying logarithm on the above equations yields:

$${v}_{S} ( i , j ) = p ( {x}_{i} , {y}_{j} ) + max \{ \begin{matrix}log ( 1 - 2 δ ) + {v}_{S} ( i - 1 , j - 1 ) \\ log ( 1 - ε ) + {v}_{I} ( i - 1 , j - 1 ) \\ log ( 1 - ε ) + {v}_{D} ( i - 1 , j - 1 )\end{matrix}$$

$${v}_{I} ( i , j ) = p ( {x}_{i} , η ) + max \{ \begin{matrix}log ( δ ) + {v}_{S} ( i - 1 , j ) \\ log ( ε ) + {v}_{D} ( i - 1 , j )\end{matrix} , and$$

$${v}_{D} ( i , j ) = p ( η , {y}_{j} ) + max \{ \begin{matrix}log ( δ ) + {v}_{S} ( i , j - 1 ) \\ log ( ε ) + {v}_{D} ( i , j - 1 )\end{matrix}$$

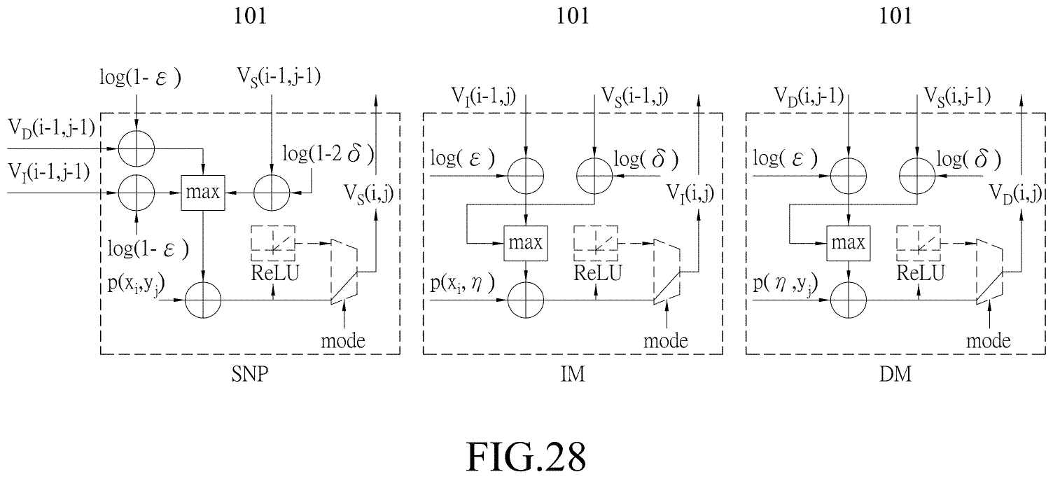

which render the calculation into a number of additions, and thus can be processed by the dynamic processing engine 10. FIG. 28 illustrates three exemplary operating units 101 that are configured to perform the above calculations, and to output the corresponding likelihoods.

Afterward, for each pair of an i^{th} character of the sequence x and a j^{th} character of the sequence y, three separate likelihoods may be obtained. The likelihoods may then be arranged into a result matrix for determining a most likely source of variant (by, for example, using one of the three likelihoods with the largest value) for subsequent analysis. The result matrix indicates, for each of the variants contained in the to-be-tested DNA sequence, a location and a most-likely type of a variant.

In use, the result matrix may be reviewed by personnel to verify the determination done by the data processing system 100, such as whether a determination of a variant is correct or erroneous. It is noted that the result matrix may also be converted to other file formats for facilitating storage and sharing.

As such, the data processing system 100 as described above is configured to process the gene sequencing data as described above using the four different modes, so as to output the result matrix to be utilized by various personnel (e.g., researchers in medical facilities, academic facilities, etc.) for different applications.

In some embodiments, the functional blocks of the data processing system 100 may be designed and integrated in the form of an integrated circuit (IC), such as a system on a chip (SoC). The SoC may also be coupled to other customized controlling circuits and/or interfaces so as to receive the gene sequencing data from an external source (e.g., a secure digital (SD) card or other storage devices), and to output the result matrix to be stored in the external source.

To sum up, embodiments of the disclosure provide a data processing system that is configured to process the gene sequencing data with the following effects.

In the preprocessing mode, the string generating module 3 generates a number (N) of partial strings from the suffix strings, respectively. Specifically, each of the partial strings includes the first to Kth characters of the corresponding one of the suffix strings. Since K<N, the amount of data to be stored in the memory device 1 for subsequent processing may be significantly reduced.

In the short-read mapping mode, the location generating module 9 is configured to first divide each of the short-reads stored in the memory device 1 into a plurality of seeds. Then, for each of the seeds thus acquired as a result of the division, the location generating module 9 determines at least one candidate row address that is associated with a candidate index of the seed in the to-be-tested DNA sequence, based on the data structure or the partial data structure. Such operations may be referred to as exact match. Then, a similarity score matrix may be constructed, and the resulting similarity score may be used to obtain a mapping location. Such operations may be referred to as inexact match.

Using the operations as described in the short-read mapping mode, the amount of calculations (i.e., candidates) may be reduced since a range of data that is to be calculated as inexact match becomes smaller. As a result, the processing time of the operations as described in the method may be significantly reduced compared with the conventional method, which may involve billions of inexact match operations. Using the operations as described in the short-read mapping mode, the number of calculations needed may be reduced from 3 billion to 100-1000.

The sorting engine 6 may be easily configured to perform operations in different stages and modes (e.g., in the preprocessing mode and in the sequence assembly mode). Additionally, by providing a large number of sorting units 61, the associated calculations may be completed with higher efficiency.

The dynamic processing engine 10 may be easily configured to perform operations in different stages and modes. Additionally, by implementing additions instead of multiplications, the corresponding design of the SoC may be made significantly simpler (i.e., one-dimensional structure instead of two-dimensional structure) and therefore smaller in size. As such, the design for the data processing system may facilitate the processing of gene sequencing data, which may involve billions of characters.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A data processing system for processing gene sequencing data, the gene sequencing data including a reference DNA sequence, a plurality of suffix strings, a plurality of indices and a plurality of short-reads, the reference DNA sequence including characters that represent nitrogen-containing nucleobases, the suffix strings being associated with a reference sequence that includes the reference DNA sequence, each of the indices indicating a location of the ending character in the reference sequence and being assigned to a corresponding one of the suffix strings, the short-reads being extracted from a to-be-tested DNA sequence, the data processing system comprising:
a string generating module;
an encoding module that is coupled to said string generating module;
a string selecting module that is coupled to said encoding module;
a sorting engine that is coupled to said encoding module and said string selecting module;
a suffix string array generating module that is coupled to said sorting engine;
a data structure generation module that is coupled to said suffix string array generating module;
a location generating module;
a dynamic processing engine that is coupled to said location generating module;
a mapping module that is coupled to said dynamic processing engine and said sorting engine; and
a variant calling module that is coupled to said dynamic processing engine;
wherein the data processing system is configured to operate in one of the following modes:
a preprocessing mode, in which
said string generating module is configured to generate a number (N) of partial strings from the suffix strings, respectively, each of the partial strings including first to Kth characters of the respective one of the suffix strings, N being a positive integer greater than 2 and K being a positive integer greater than 2, and N>K,
said encoding module is configured to use binary values to encode the partial strings to generate a number (N) of encoded partial strings, to encode the short-reads to generate a plurality of to-be-tested encoded strings, and to encode the reference DNA sequence to generate a reference encoded string,
said string selecting module is configured to select a number (P*Q) of the encoded partial strings using an upsampling process, and said sorting engine is configured to perform a sorting operation on the number (P*Q) of the encoded partial strings to sort the encoded partial strings in an ascending order, and said string selecting module is configured to select, using a downsampling process, a number (P) of the encoded partial strings from the number (P*Q) of the encoded partial strings that have been sorted as separation strings, wherein P and Q are integers,
said sorting engine is configured to perform a grouping operation on the number (N) of the encoded partial strings, using the number (P) of the separation strings, to sort the encoded partial strings into a number (P+1) of groups, and to perform a sorting operation on the encoded partial strings included in each of the number (P+1) of groups, so as to obtain a sorted list of the number (N) of the encoded partial strings,
said suffix string array generating module is configured to generate a suffix string array based on the sorted list of the number (N) of the encoded partial strings, and
said data structure generation module is configured to generate, based on the suffix string array and the associated indices, a data structure associated with the reference DNA sequence, the data structure including a CNT table, an SA table, an F table, an L table and an OCC table, the F table including a column that lists the first characters of the suffix strings included in rows of the suffix string array, the L table including a column that lists the last characters of the suffix strings included in the rows of the suffix string array, the SA table including a column that lists the indices associated with of the suffix strings included in the rows of the suffix string array, the CNT table including a column that lists, for each of the characters, a row address of a prior row immediately before a row at which the character first appears, the OCC table including columns that correspond respectively to the characters and that each list cumulative numbers of appearances of the corresponding one of the characters in the rows of the L table,
a short-read mapping mode, in which
said location generating module is configured to divide each of the short-reads into a plurality of seeds, and, for each of the seeds thus acquired as a result of the division, determine, based on the data structure, at least one candidate row address that is associated with a candidate index indicating a position of the seed in the to-be-tested DNA sequence,
said dynamic processing engine is configured to implement a similarity algorithm with respect to each of the short-reads and the content included in the part of the reference DNA sequence that is indicated by the candidate indices associated with the seeds of the short-reads, so as to obtain a similarity score for the short-read, and
said mapping module is configured to, for each of the short-reads, determine, based on the similarity score, a mapping location for the short-read,
a sequence assembly mode, in which said sorting engine is configured to construct an encoded assembled sequence based on the to-be-tested encoded strings and the reference encoded string and the mapping locations for the short-reads, the encoded assembled sequence indicating a haplotype sequence, and
a variant calling mode, in which
said dynamic processing engine is configured to perform the similarity algorithm with respect to the haplotype sequence and the reference DNA sequence, and
said variant calling module is configured to evaluate a location and a type of a variant in the haplotype sequence based on the result of the similarity algorithm.

2. The data processing system of claim 1, further comprising a memory device that is coupled to said encoding module, said string selecting module, said sorting engine and said dynamic processing engine, and that is configured to store the gene sequencing data and other information that is generated during operations of the data processing system.

3. The data processing system of claim 2, wherein:
in the preprocessing mode, said sorting module is configured to use the number (P) of the separation strings stored in said memory device to sort the encoded partial strings into the number (P+1) of groups.

4. The data processing system of claim 2, further comprising a suffix string generating module that is coupled to said memory device, and that is configured to generate the number (N) of suffix strings and to assign an index to each of the suffix strings.

5. The data processing system of claim 2, wherein:
said data structure generation module is coupled to said memory device so as to store the data structure generated by said data structure generation module therein;
said location generating module is coupled to said memory device so as to access said memory device to obtain the data structure for implementing the determination of the at least one candidate row address.

6. The data processing system of claim 2, wherein:
said data structure generation module is configured to generate a partial data structure based on the data structure, and is coupled to said memory device so as to store the partial data structure therein;
said location generating module is coupled to said memory device so as to access said memory device to obtain the partial data structure, and is configured to reconstruct the data structure based on the partial data structure before implementing the determination of the at least one candidate row address.

7. The data processing system of claim 6, wherein said sorting engine includes a plurality of sorting units that are arranged in a plurality of series connections, and each of said sorting units includes:
a first data input node for receiving a data signal from other parts of the data processing system;
a second data input node for receiving data from a preceding one of the sorting units that is connected in front of the sorting unit in the same series connection;
a first control input node for receiving a first control signal from the preceding one of the sorting units;
a second control input node for receiving a second control signal from an external source;
a first output node for transmitting data to a succeeding one of the sorting units that is connected behind of the sorting unit in the same series connection;
a second output node for transmitting the first control signal to the succeeding one of the sorting units;
a third output node;
a fourth output node;
a register that includes a clock input node, a data input node, and a data output node that is connected to the first output node of said sorting unit;
a comparator that includes two input nodes connected to the first data input node and the data output node of said register, respectively, and an output node connected to the third output node and the second output node of said sorting unit;
a first 2*1 multiplexor (MUX) that includes a first input node connected to the first data input node of said sorting unit, a second input node connected to the second data input node of said sorting unit, a control node connected to the first control input node of said sorting unit, and an output node;
a 3*1 MUX that includes a first input node connected to the first output node of the preceding sorting unit, a second input node connected to the output node of said first 2*1 MUX, a third input node connected to the first output node the succeeding sorting unit, a control node connected to the second control input node of said sorting unit, and an output node;
a second 2*1 MUX that includes a first input node connected to the output node of said 3*1 MUX, a second input node connected to the output node of said register, a control node connected to the output node of said comparator, and an output node connected to the input node of said register;
an inverter that is connected to the first control input node of said sorting unit; and
an AND gate that includes two input nodes connected to said inverter and the output node of said comparator, respectively, and an output node connected to the fourth output node of said sorting unit.

8. The data processing system of claim 7, wherein:
said sorting engine further includes an adder that includes a plurality of input nodes that are connected respectively to the third output nodes respectively of said sorting units, and an output node;
in the preprocessing mode, each of the number (P) of the separation strings is stored in one of the sorting units, each of the number (N) of the encoded partial strings is fed into the sorting units via the first data input nodes respectively of said sorting units, and each of the encoded partial strings is grouped into one of the number (P+1) of groups based on a resulting output of said adder.

9. The data processing system of claim 7, wherein after each of the number (N) of the encoded partial strings has been grouped, said sorting engine is configured to perform the sorting operation on the encoded partial strings included in each of the number (P+1) of groups, so as to obtain the sorted list of the number (N) of the encoded partial strings.

10. The data processing system of claim 7, wherein, in the sequence assembly mode:
said sorting units are controlled to store data of a reference encoded sub-sequence that corresponds with a read with consecutive same characters and with a largest binary value;
the first data input nodes of said sorting units are configured to receive a plurality of encoded sub-sequences that are associated with consecutive same characters included in one of the to-be-tested encoded string which is encoded from the short-reads and the reference encoded string which is encoded from the reference DNA sequence, and the encoded sub-sequences are stored in the corresponding ones of said sorting units, so as to create a De Bruijn graph;
an encoded sub-string that is associated with first to kth characters of one of the short-reads with a smallest mapping location is used as an input to the first data input nodes of each of said sorting units, where k is an integer
said sorting units are configured to compare the binary values of the encoded sub-sequence and the encoded sub-string, resulting in the data stored in the one of the sorting units being outputted for reassembly of the encoded string that corresponds with the short-read; and
the data processing system is configured to repeat the above operations until the encoded string that corresponds with the short-read is assembled, and store the encoded string in said memory device.

11. The data processing system of claim 2, wherein:
said dynamic processing engine includes a plurality of operating units that are configured to perform the similarity algorithm, wherein the similarity algorithm is a Smith-Waterman algorithm;
each of the operating units includes three input nodes, and an output node for outputting an output signal, and each of the three input nodes is connected to the output node of another one of the operating units;
in the short-read mapping mode, said dynamic processing engine is configured to implement the similarity algorithm with respect to each of the short-reads and the content included in the part of the reference DNA sequence, so as to obtain a scoring matrix, and a highest score included in the scoring matrix is used as the similarity score associated with the short-read and the candidate index; and
in the variant calling mode, said dynamic processing engine is configured to perform the similarity algorithm with respect to each of the haplotype sequences and the reference DNA sequence, so as to obtain a similarity score matrix and a scoring direction matrix that contains information related to the similarity score matrix.


SEQUENCE LISTING (7 sequences)

SEQ ID NO: 1 - DNA, 10 bp, synthetic construct
  source 1..10: mol_type = other DNA
catgaaagga

SEQ ID NO: 2 - DNA, 40 bp, synthetic construct
  source 1..40: mol_type = other DNA
catgaaaggagacgaaaggatttgataccacaacatttgg

SEQ ID NO: 3 - DNA, 16 bp, synthetic construct
  source 1..16: mol_type = other DNA
acgaaaggatttgata

SEQ ID NO: 4 - DNA, 11 bp, synthetic construct
  source 1..11: mol_type = other DNA
gataccacagc

SEQ ID NO: 5 - DNA, 11 bp, synthetic construct
  source 1..11: mol_type = other DNA
aaggtgacgaa

SEQ ID NO: 6 - DNA, 10 bp, synthetic construct
  source 1..10: mol_type = other DNA
cagcatttgg

SEQ ID NO: 7 - DNA, 16 bp, synthetic construct
  source 1..16: mol_type = other DNA
catgaaaggtgacgaa